SEQUENCE LISTING (2 sequences)

SEQ ID NO: 1 - DNA, 19 bp, Bacterial
agagttgatcatggctcag

SEQ ID NO: 2 - DNA, 22 bp, Bacterial
tacggytaccttgttacgactt

(19) United States
(12) Patent Application Publication
(10) Pub. No.: US 2006/0110456 A1
(43) Pub. Date: May 25, 2006

(54) Method for biocidal and/or biostatic treatment and compositions therefor

(75) Inventors: Serena Lay Ming Teo, Singapore (SG); Amy Mei Fun Choong, Singapore (SG); Tsai Min Sin, Singapore (SG); Daniel Rittschof, Morehead City, NC (US); James Scott Maki, Racine, WI (US)

Correspondence Address: CONLEY ROSE, P.C., P. O. BOX 3267, HOUSTON, TX, 77253-3267, US

(73) Assignee: NATIONAL UNIVERSITY OF SINGAPORE, Singapore (SG)

(21) Appl. No.: 11/265,833

(22) Filed: Nov. 3, 2005

Related U.S. Application Data
(60) Provisional application No. 60/624,649, filed on Nov. 3, 2004.

(51) Int. Cl. A61K 9/24

(52) U.S. Cl. 424/472

(57) ABSTRACT

There is provided a method of biocidal and/or biostatic treatment and compounds and compositions thereof. In particular, there is provided a composition comprising at least one compound having the formula R¹R²NR³, wherein R¹, R² and R³, are selected from the group consisting of hydrido, alkyl, aryl, heteroaryl, cycloalkyl, cycloaryl, cycloheteroaryl, alkenyl and alkynyl. In particular, the compounds and compositions of the invention are suitable for preventing, reducing and/or eliminating biofilms and/or fouling.

### FIELD OF INVENTION

The present invention relates to methods for biocidal and/or biostatic treatment and to compositions therefor. In particular, the invention relates to products and compositions suitable in a method for preventing, reducing and/or eliminating biofilms and/or fouling of structures by microorganisms and other organisms.

### BACKGROUND OF THE INVENTION

Whenever a surface or substrate is exposed to, or immersed in a liquid such as water, organisms will attach or adhere to the surface. An example of this phenomenon is that of barnacles attaching and growing on the hull of a ship. This presents a problem as fouling increases the drag of the ship moving through the water and consequently increases fuel consumption.

Another example is that of bacteria, fungi or algae forming biofilms in containers of standing water or in re-circulated water used in water fountains, reproducing to reach unhealthy levels in the water. These phenomena are undesirable and present a problem. In the past, chemical solutions to prevent, reduce or eliminate the attachment and growth of these fouling microorganisms and small organisms have been provided but these solutions carry with them undesirable side effects and environmental problems.

Fouling of surfaces in the marine environment is a multi-level process which involves passive attachment of molecular and marine snow fouling, as well as active attachment by micro- and macro-organisms (Clare et al 1992). Bacteria have the most numerous propagules in aquatic environments and are thus, often observed as the earliest colonizers of new surfaces placed into water. Once attached, they can modify the substrata and act as associative cues to attract or repel macrofouling organisms. Molecules that especially impact microbial and macro-fouling, have potential application as antifoulants.

Existing commercial solutions are antifouling or foul-release. Antifouling uses broad-spectrum biocides which kill foulers by virtue of oxidation or, more usually, toxic metal ions. Foul-release coatings are mainly silicon based polymers that are easy to clean, and best of these usually also contain additives and catalysts that kill organisms. Legislation and agreements due to environmentally unacceptable consequences of toxic antifouling agents in coatings such as TBT, have prompted interest to develop new less environmentally pernicious antifoulants. Antifouling compounds obtained from marine “natural products” are common in the academic literature, but the development of commercial coatings using natural products is blocked by cost, technical issues related to synthesis and compatibility with existing coatings, time required to meet government environmental registration and performance standards in which coatings are expected to perform as well as existing toxic coatings with unacceptable environmental impacts (Rittschof, 2000). Clare (1996) listed over 50 core natural product structures with potential antifouling activity from published research, and approximately another 100 more have been added since then (Rittschof, 2001). Thus, although natural products for antifouling use have been disclosed, the development of functional coatings based upon natural products involves technological, financial, temporal and regulatory difficulties.

The goal of another paper (Rittschof et al, 2002) was to gather preliminary data aimed to the construction of additional working hypotheses to screen for antifouling compunds. However, Rittschof et al (2002) conceded that the lack of consistency between toxicity and settlement data suggested that an approach based on the biological potency of pharmaceuticals in vertebrates had only limited promise. Accordingly, Rittschof et al. (2002) did not prove or provide reliable data that any of the tested drugs can be used as antifoulings.

Teo et al (2003) also relates to preliminary data on the hypothesis of considering pharmaceuticals as antifoulings. However, several drugs showed an undesirable marked toxicity to Mysid shrimps. Other drugs showed low effect of fertilization. Accordingly, no indication of use of any of these drugs as antifoulings can be deducted from Teo et al (2003).

All living organisms have common basic biochemical pathways. Animals share a huge variety signalling, metabolic, and control pathways. It is likely that existing commercial antifouling compounds and novel compounds found to have antifouling effects, act on one or more of these pathways. However, the chemical complexity of the pathways, the multitude of compounds and the ancillary scientific, technical and societal issues require novel solutions not presently in the art. There is a need in this field of the art, of a new approach in order to provide alternative compounds and methods for preventing, reducing or eliminating fouling and/or biofilm formation.

### SUMMARY OF THE INVENTION

The present invention addresses the problems of the prior art and provides new compositions and methods for biocidal and/or biostatic treatment using existing pharmaceuticals, with their known synthesis and biochemistry, for development as antifoulants. In particular, the invention provides products and/or compositions suitable in a method of preventing, reducing and/or eliminating biofilms and/or fouling.

According to one aspect, there is provided a biocidal and/or biostatic composition. In particular, there is provided a composition comprising at least one compound having the formula R¹R²NR³, wherein R¹, R² and R³ are selected from the group consisting of hydrido, alkyl, aryl, heteroaryl, cycloalkyl, cycloaryl, cycloheteroaryl, alkenyl and alkynyl.

At least one compound may be a substituted amine wherein R¹ and R² are selected from the group consisting of: alkyl, heteroaryl, cycloalkyl, cycloaryl, cycloheteroalkyl, and cycloheteroaryl; and R³ is alkyl.

R¹ and R² may form part of a carbocyclic or heterocyclic ring; and wherein R³ is alkyl. R³ is may be methyl, ethyl, propyl or butyl.

In particular, the composition of the present invention comprises at least one compound selected from the group consisting of: cyclizine, haloperidol decanoate, loperamide, prochlorperazine, trifluoperazine, fluoxetine, ondansetron, sertraline hydrochloride, olanzapine and salts of these compounds.

The composition of the present invention may be useful for prevention of fouling and/or biofilm formation.

The composition according to the invention, may further comprise mixing at least one binder with at least one compound. The binder may be a binder suitable for biocidal and/or biostatic treatment. For example, a binder suitable in a treatment for the prevention of fouling and/or biofilm formation. In particular, the binder may be a curable binder to form a mixture before applying the composition to a medium.

There is also provided a method for biocidal and/or biostatic treatment of a medium comprising: (i) providing at least one compound or a composition comprising at least one compound, the compound having the formula R¹R²NR³, wherein R¹, R² and R³ are selected from the group consisting of: hydrido, alkyl, aryl, heteroaryl, cycloalkyl, cycloaryl, cycloheteroaryl, alkenyl and alkynyl; and (ii) applying the compound to the medium.

In particular, the compound having formula R¹R²NR³ may be any one of the compounds according to the present invention. The medium may be a solid medium. The method of the present invention may further comprise mixing the compound with a binder, for example, a curable binder to form a mixture before applying the composition to the medium, preferably a solid medium, and then allowing the mixture to cure. The medium may also be a liquid, for example an aqueous liquid. More in particular, water. The liquid may be contained in a container.

The binder according to the present invention may comprise at least one anti-fouling compound at less than standard concentration.

The method of the present invention may be a treatment to prevent of fouling and/or biofilm formation of a medium.

There is also provided a coated substrate wherein the coating comprises at least one compound having the formula R¹R²NR³, wherein R¹, R² and R³ are selected from the group consisting of hydrido, alkyl, aryl, heteroaryl, cycloalkyl, cycloaryl, cycloheteroaryl, alkenyl and alkynyl.

The coated substrate may be any substrate which may be treated with any compound or composition according to the present invention. For example, the substrate may be any substrate exposed or in contact with a liquid, such as water. For example, a portion of or an entire vessel. The substrate may also be, for example, a container or portion thereof of standing water. The compounds or compositions according to the invention may also be used to treat re-circulated liquid, such as re-circulated water of fountains.

### BRIEF DESCRIPTION OF THE FIGURES AND REFERENCE TO SEQUENCE LISTINGS

FIGS. 1 and 2. Structure of active ingredients of compounds of the present invention.

FIG. 3. Growth assay: 24-hour change in absorbance for 10 strains of bacteria in the presence of ZLT. Of the ten strains tested, only strain number 14 grew in the presence of ZLT. In two of three trials, ZLT promoted growth as measured by increase in absorbance. Growth was entirely inhibited in all trials for the other nine strains tested.

FIG. 4. Growth assay: 24-hour change in absorbance for ten strains of bacteria in the presence of PZC. Of the ten strains tested, only strain numbers 1 and 14 grew in the presence of PZC. In these two strains PZC promoted growth as measured by increase in absorbance. Growth was entirely inhibited in all trials for the other eight strains tested.

FIG. 5. Growth assay: 24-hour change in absorbance for ten strains of bacteria in the presence of TFZ. Of the ten strains tested, only strain numbers 1 and 14 grew in the presence of TFZ. TFZ promoted growth as measured by increase in absorbance in one trial out of three for strain 1 and two trials out of three for strain 14. Growth was entirely inhibited in all trials for the other strains tested.

FIG. 6. Growth assay: 24-hour change in absorbance for ten strains of bacteria in the presence of PCZ. Of the ten strains tested, only strain numbers 1 and 14 grew in the presence of PCZ. PCZ promoted growth as measured by increase in absorbance in one trial out of three for strain 1 and two trials out of three for strain 14. Growth was entirely inhibited in all trials for the other eight strains tested.

FIG. 7. Growth response of bacteria strain 28 in the presence and absence of PZC (25 μg ml−1).

FIG. 8. Example of slight growth response of bacteria strain 11 in presence of PCZ (25 μg ml−1).

FIG. 9. Recovery of growth by strain 16 in presence of ZLT (25 μg ml−1).

FIG. 10. Little or no growth response by bacteria strain 28 to ZLT (25 μg ml−1)

FIG. 11. Results of settlement bioassay for ZPX. Mortality of cyprids in the wells containing ZPX was not very different from the control. Cyprids that did not settle remained swimming. ZPX prevented the settlement of cyprids.

FIG. 12. Percentage cover of fouling on ZLT, PCZ, HDL, ZPX, ZFN and IMD coated rods.

FIG. 13. Percentage cover for CYZ and PZC coated rods.

FIG. 14. Adhesion of bacterial cells to tissue-culture treated polystyrene (NTC) and untreated polystyrene (TC) microplates.

FIG. 15. SHM data for application of drugs to NTC and TC plates.

FIG. 16. Effect of CYZ on formation of bacterial biofilms on NTC and TC plates. The black bars give a measure of the biofilm formed in the control wells (bacteria only), and the grey bar is the biofilm obtained in the presence of 25 ug ml−1 of drug. No significant reduction in biofilm formation was observed for CYZ.

FIG. 17. Effect of HDL on formation of bacterial biofilms on NTC and TC plates. The black bars give a measure of the biofilm formed in the control wells (bacteria only), and the grey bar is the biofilm obtained in the presence of 25 ug ml−1 of drug. No significant reduction in biofilm formation was observed for most strains, except for strain 28, where there appears to be a slight effect (0.05<p<0.1).

FIG. 18. Effect of IMD on formation of bacterial biofilms on NTC and TC plates. The black bars give a measure of the biofilm formed in the control wells (bacteria only), and the grey bar is the biofilm obtained in the presence of 25 ug ml−1 of drug. There was reduction of biofilm growth in strain 28 on NTC but not on TC. There appears to be a slight effect on strain 4 and 14 (on TC) but this was not marked.

FIG. 19. Effect of ZFN on formation of bacterial biofilms on NTC and TC plates. The black bars give a measure of the biofilm formed in the control wells (bacteria only), and the grey bar is the biofilm obtained in the presence of 25 ug ml−1 of drug. There was a marked reduction of biofilm growth for most on NTC but not on TC.

FIG. 20. Effect of ZPX on formation of bacterial biofilms on NTC and TC plates. The black bars give a measure of the biofilm formed in the control wells (bacteria only), and the grey bar is the biofilm obtained in the presence of 25 ug ml−1 of drug. There was a marked reduction of biofilm growth for all strains on NTC, but only a slight reduction for strain 4 on TC.

FIG. 21. Effect of PZC on formation of bacterial biofilms on NTC and TC plates. The black bars give a measure of the biofilm formed in the control wells (bacteria only), and the grey bar is the biofilm obtained in the presence of 25 ug ml−1 of drug. PZC showed a slight effect on strain 4 (p<0.1).

FIG. 22. Effect of ZLT on formation of bacterial biofilms on NTC and TC plates. The black bars give a measure of the biofilm formed in the control wells (bacteria only), and the grey bar is the biofilm obtained in the presence of 25 ug ml−1 of drug. There was a reduction of biofilm growth for most strains (except strain 14) on NTC, but only for strain 28 on TC.

FIG. 23. Effect of PCZ on formation of bacterial biofilms on NTC and TC plates. The black bars give a measure of the biofilm formed in the control wells (bacteria only), and the grey bar is the biofilm obtained in the presence of 25 ug ml−1 of drug. PCZ was active against strain 4, and a slight effect on strain 27.

FIG. 24. Effect of TFZ on formation of bacterial biofilms on NTC and TC plates. The black bars give a measure of the biofilm formed in the control wells (bacteria only), and the grey bar is the biofilm obtained in the presence of 25 ug ml−1 of drug. TFZ was active against strain 4, 14, 27 on NTC but no reduction of biofilms was observed on the TC plates.

FIG. 25. Effect of 25 ug ml−1 ZPX on established biofilm. The first bar shows the bacteria film that forms in the absence of drug. The second bar reflects the bacteria film left after treatment with ZPX.

FIG. 26. Effect of 25 ug ml−1 ZFN on established biofilms.

FIG. 27. Effect of ZPX on bacteria at 50 ug ml−1. The first bar gives the biofilm growth in absence of ZPX. The second bar is the biofilm that forms in the presence of ZPX (=ZPX and bacteria added at the same time), whilst the third bar shows the biofilm left after it has been treated with ZPX (bacteria added first, then ZPX added to biofilm).

FIG. 28. Effect of ZFN on bacteria at 50 ug ml−1.

FIG. 29. Effect of IMD (tablet form) on settlement of cyprids on glass and plastic.

FIG. 30. Effect of IMD (pure compound) on settlement of cyprids on glass and plastic.

FIG. 31. Settlement of cyprids on plastic: effect of drug form.

FIG. 32. Settlement of cyprids on plastic: effect of solvent.

FIG. 33. Effect of ZPX on cyprid settlement on different substrata.

FIG. 34. Effect of UV on drug toxicity to barnacle nauplii.

FIG. 35. Stability of drugs in seawater under light/dark conditions over 4 weeks.

FIG. 36. Fouling cover on IMD treated rods.

FIG. 37. Fouling cover on ZPX treated rods.

FIG. 38. Fouling cover on ZFN treated rods.

FIG. 39. Fouling cover on ZFN+ZPX treated rods.

FIG. 40. Fouling cover on IMD−ZFN treated rods.

FIG. 41. Fouling cover on IMD−ZPX treated rods.

FIG. 42. General structure of the compounds of the present invention.

### SEQUENCE LISTINGS

PCR Primer Sequences:

| | | |
|---|---|---|
| 27F: | 5′ AGAGTTGATCATGGCTCAG 3′ | (SEQ ID NO:1) |
| 1492R: | 5′ TACGGYTACCTTGTTACGACTT 3′ | (SEQ ID NO:2) |

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

Definitions

As used herein, the term “fouling” refers to the attachment and growth of microorganisms and small organisms to a substrate exposed to, or immersed in, a liquid medium, for example an aqueous medium, as well as to an increase in number of the microorganisms and/or small organisms in a container of the liquid medium. Accordingly “foulers” or “microfoulers” are used interchangeably and refer to the organisms that foul a substrate. Fouling may occur in structures exposed to or immersed in fresh water as well as in sea water. In particular, the term may be used to refer to a solid medium or substrate exposed to, or immersed in sea water. Accordingly, the term “antifouling” refers to the effect of preventing, reducing and/or eliminating fouling. Antifouling agents or compounds are also called “antifoulants”.

An antifoulant compound is usually applied at a standard concentration which is the concentration that is effective for its purpose. Accordingly, a concentration less than or below the standard concentration is one where the antifoulant is not effective when it is used alone.

The term “substrate” as used herein refers to a solid medium such as surfaces of structures or vessels exposed to, or immersed in a liquid medium. The liquid medium may be fresh water or seawater and may be a body of water in a manmade container such as a bottle, pool or tank, or the liquid may be uncontained by any manmade container such as seawater in the open sea.

A “structure” as used herein refers to natural geological or manmade structures such as piers or oil rigs and the term “vessel” refers to manmade vehicles used in water such as boats and ships.

The “microorganisms” referred to herein include viruses, bacteria, fungi, algae and protozoans. “Small organisms” referred to herein can include organisms that commonly foul substrates exposed to, or immersed in, fresh water or seawater such as crustaceans, bryozoans and molluscs, particularly those that adhere to a substrate. Examples of such small organisms include barnacles and mussels and their larvae. Small organisms can also be called small animals.

Further, the term “microfouling” refers to fouling by microorganisms and the term “macrofouling” refers to fouling by organisms larger than microorganisms such as small organisms defined above.

The terms “biocide” or “biocidal compound” refer to compounds that inhibit the growth of microorganisms and small organisms by killing them. The terms “biostatic” or “biostatic compound” refer to compounds that inhibit the growth of microorganisms or small organisms by preventing them from reproducing and not necessarily by killing them. The term “growth” as used herein refers to both the increase in number of microorganisms and small organisms, as well to the development of a small organism from juvenile to adult stages. Accordingly, biocides and biostatics can be applied as a treatment to a body of liquid or to a substrate surface to inhibit the growth of microorganisms and small organisms. As such, biocides and biostatics can be antifoulants and can prevent, reduce or eliminate biofilm formation. Accordingly, the terms “bacteriocidal” and “bacteriostatic” refer to effects of compounds on bacteria.

The term “bioactivity” as used herein refers to the effect of a given agent or compound, such as a biocidal or biostatic compound, on a living organism, particularly on microorganisms or small organisms.

A “biofilm” is a complex aggregation of microorganisms, usually bacteria or fungi, marked by the excretion of a protective and adhesive matrix. Biofilms are also often characterized by surface attachment, structural heterogeneity, genetic diversity, complex community interactions, and an extracellular matrix of polymeric substances. Biofilms may also be more resistant to antibiotics compared to unaggregated bacteria due to the presence of the matrix.

The term “pharmaceutical” as it relates to a use, agent, compound or composition, refers to the medical treatment of a disease or disorder in humans or animals. Accordingly, a pharmaceutical compound is a compound used for the medical treatment of a disease or disorder in humans or animals.

As used herein, the term “standard concentration” as it pertains to an anti-fouling agent or compound, refers to the concentration at which the agent or compound is effective against microorganisms or small organisms at which it are directed when that agent or compound is used alone. Accordingly, the term “effective” means having a desired effect and the term “below standard concentration” refers to the level at which the agent or compound is not effective when used alone.

The term “pharmaceutically acceptable salt” of a compound as used herein means a salt that is pharmaceutically acceptable and that possesses the desired pharmacological activity of the parent compound.

As the compounds of the present invention can also have pharmaceutical activity, they are also referred to herein as “pharmaceutical compounds” or simply as “pharmaceuticals”.

Description

The present invention provides biocidal and/or biostatic compound(s) or new composition comprising at least one said compound(s) having biocidal and/or biostatic effect. In particular, the invention provides new compounds or a new use for compound(s) already known in the art, as well as new composition comprising any new or known compound as herein described. In particular, the invention provides compound(s) and or composition(s) having biocidal and/or biostatic effects on microorganisms and/or small organisms that form biofilms and/or foul a structure. In particular, the compound according to the invention is a substituted or unsubstituted amine. In particular, the compound of the invention may be described by the general formula for a substituted or unsubstituted amine:
R¹R²NR³
wherein each of R¹, R² and R³, independently, are selected from the group consisting of: hydrido, substituted or unsubstituted groups such as alkyl, aryl, heteroaryl, cycloalkyl, cycloheteroakyl, cycloaryl, cycloheteroaryl, alkenyl, alkynyl, and combinations thereof. However, R¹, R² and R³ are not limited to the substituents listed above. For example, it will be evident to a skilled person in the field how to choose one ore more substituents not specifically listed above suitable for the purpose of the present invention. For example, a skilled person may contemplate derivative of the substituents listed above suitable for the purpose of the present invention. In particular, there is provided a biocidal and/or biostatic composition comprising said compound.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. The substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Examples of substituents include, for example, halogen, alkyl, cycloalkyl, aryl, hydroxyl, amino, nitro, sulfhydryl, hydroxy, amino, carbonyl, carboxyl, silyl, ether, alkylthio, sulfonyl, heterocyclyl, trifluoromethyl, cyano, and the like.

The biocidal and/or biostatic compound, preferably comprised in a composition, according to the invention may be a compound having formula R¹R²NR³, wherein at least one compound is a substituted amine wherein R¹ and R² are selected from the group consisting of alkyl, heteroaryl, cycloalkyl, cycloaryl, cycloheteroalkyl, and cycloheteroaryl; and R³ may be selected from the group consisting of: hydrido, substituted or unsubstituted groups such as alkyl, aryl, heteroaryl, cycloalkyl, cycloheteroakyl, cycloaryl, cycloheteroaryl, alkenyl, alkynyl, and combinations thereof. R³ may be alkyl. For example, wherein R³ may be selected from the group consisting of: methyl, ethyl, propyl and butyl. R¹ and R² may form part of a carbocyclic or heterocyclic ring wherein the heterocyclic atom may be nitrogen, oxygen or sulphur, and R³ is above defined. Alternatively, R¹ and R² may form a part of a carbocyclic or heterocyclic ring wherein the heterocyclic atom may be nitrogen, oxygen or sulphur. R¹ may be linked to R² to form a ring that has a ring size of 4-7. R¹ may be linked to R² to form a ring containing nitrogen heteroatoms. R¹ may be linked to R² to form a six-membered ring containing one or more nitrogen atoms.

In particular, the compound of the invention, or the compound comprised in a composition, is compound of formula R¹R²NR³, wherein R¹ and R² is alkyl, heteroaryl, cycloalkyl, cycloaryl, cycloheteroalkyl, cycloheteroaryl; and wherein R³ is alkyl. In the compound of the invention, R¹ and R² may form part of a carbocyclic or heterocyclic ring; and R³ is alkyl. In particular, R¹ and R² may form part of a carbocyclic or heterocyclic ring; and R³ is selected from the group consisting of: methyl, ethyl, propyl and butyl.

The compound of the invention may be selected from the group consisting of: cyclizine, haloperidol decanoate, loperamide, prochlorperazine, trifluoperazine, fluoxetine, ondansetron, sertraline hydrochloride, olanzapine and salt(s) thereof.

These compounds are known pharmaceutical compound(s) and they are described in more details below.
(I) A compound of formula 1:

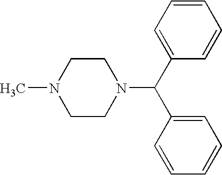

An example of a compound with this formula is cyclizine hydrochloride and a composition containing the compound is Marzine™.

Marzine™ (Pfizer), contains the active ingredient, Cyclizine hydrochloride (CYZ)

Marzine™ (Marezine™ in the USA) is a non-prescription tablet used for prevention of motion sickness. It is also used for prevention of nausea and vomiting caused by narcotic analgesics and radiotherapy. Sale of Marzine™ was discontinued in December 2003.

Pharmaceutical form: Marzine™ is supplied as a round white tablet containing 50 mg of the active ingredient, cyclizine.

Physical properties of active ingredient: Cyclizine hydrochloride is soluble in water, with a MW 302.8.

Pharmacodynamics in vertebrates: Cyclizine is a histamine H1 receptor antagonist of the piperazine class. It has anticholinergic and antiemetic properties.
(II) A compound of 2:

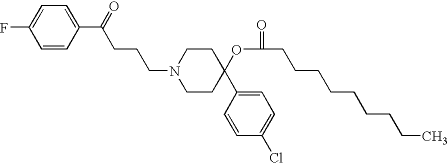

An example of a compound with this formula is haloperidol decanoate (HDL) and a composition containing the compound is Haldol®.

Haldol® (Ortho-McNeil), contains the active ingredient, haloperidol decanoate (HDL)

Haldol® is a long-acting parenteral anti-psychotic drug used in the management of patients requiring prolonged therapy (such as in cases of chronic schizophrenia).

Pharmaceutical form: Haldol® tablets are supplied as small green rounded tablets containing 5 mg of the active ingredient, haloperidol decanoate.

Physical properties of active ingredient: Haloperidol decanoate is a white solid, almost insoluble in water (0.01 mg/mL) but is soluble in most organic solvents. Haloperidol has a MW of 375.9.

Pharmacodynamics in vertebrates: Haloperidol is a dopaminergic receptor antagonist that increases dopamine release by blocking presynaptic D2 receptors. It has only a weak postsynaptic effect.
(III) A compound of formula 3:

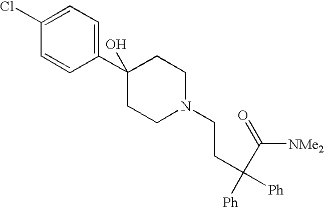

An example of a compound with this formula is loperamide hydrochloride (IMD) and a composition containing the compound is Loperamil™.

Loperamil™ contains the active ingredient, Loperamide hydrochloride (IMD)

Loperamide hydrochloride was available from several manufacturers and the form used was supplied under the tradename Loperamil™ manufactured by Drug Houses of Australia (DHA) Pte Ltd. It is indicated for the symptomatic control of acute and chronic diarrhoea.

Pharmaceutical form: Loperamil™ is supplied as a bright-blue coated tablet that is white inside, containing 2 mg of the active ingredient loperamide hydrochloride.

Physical properties of active ingredient: Loperamide hydrochloride is slightly soluble in water, and soluble in methanol, isopropyl alcohol and chloroform. It is a white-yellow powder with a MW 513.51.

Pharmacodynamics in vertebrates: Loperamide binds to opiate receptors in the gut wall, inhibiting the release of acetylcholine and prostaglandins.
(IV) A compound of 4a or 4b:

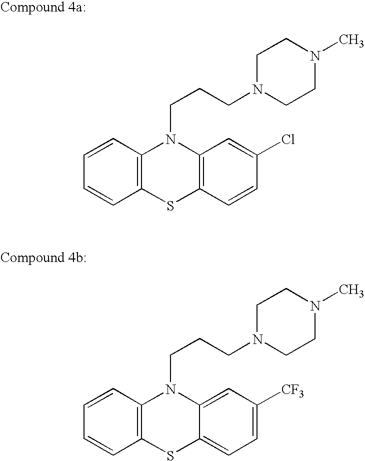

The compounds 4a and 4b are structural analogues. Compounds with the formula 4b differ from that of 4a in that they have different heteroaromatic substituents on R³.

Examples of compounds with formulae 4a and 4b are Prochlorperazine (PCZ) and Trifluoperazine (TFZ) respectively.

Generic drugs containing the active ingredients, prochlorperazine maleate (PCZ) and analogue, trifluoperazine (TFZ)

These two compounds are structural analogues and are available from several manufacturers.

Prochlorperazine is an oral and parenteral antiemetic and antipsychotic agent. It is mainly used for management of nausea and vomiting but shares many of the actions of antipsychotics. It may be prescribed for short-term treatment of generalized non-psychotic anxiety.

Pharmaceutical form: Prochlorperazine maleate was supplied as round white tablets labeled as “SMT Tablets” manufactured by Malaysia Chemist Pte Ltd. Each tablet contained 5 mg of the active ingredient, prochlorperazine maleate.

Physical properties of active ingredient: Prochlorperazine is a piperazine phenothiazine derivative, soluble in water, with a MW 606.10 (maleate form), or 373.95 (base compound).

Pharmacodynamics in vertebrates: Prochlorperazine blocks postsynaptic dopamine receptors in the mesolimbic system and increases dopamine turnover by blockade of the DA somatodendritic autoreceptor. Prochlorperazine possesses moderate anticholinergic and alpha-adrenergic receptor blocking effects.

Trifluoperazine hydrochloride is an anti-psychotic drug used to treat mental disorders.

Pharmaceutical form: Trifluoperazine was supplied as small blue round tablets containing 5 mg of active ingredient, manufactured by USP.

Physical properties of active ingredient: Trifluoperazine is a yellow solid. It has a MW 480.43 (as hydrochloride).

Pharmacodynamics in vertebrates: It is a calmodulin antagonist. It also inhibits Ca2+/calmodulin-dependent phosphodiesterase and it is a potent irreversible inhibitor of cAMP-gated cationic channels. It is known to alter the effect of dopamine in the CNS, and possess significant anti-cholinergic and alpha adrenergic blocking action.
(V) A compound with the formula 5:

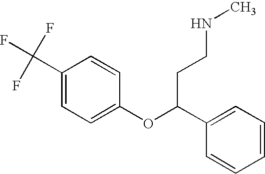

An example of a compound with this formula is fluoxetine hydrochloride (PZC) and a composition containing the compound is Magrilan™.

Magrilan™ (Medochemie Ltd—Cyprus) contains the active ingredient, fluoxetine hydrochloride (PZC)

Fluoxetine was available from several drug manufacturers. The tablets used for the experiments were traded under the brand name Magrilan™. Fluoxetine hydrochloride is an antidepressant for oral administration, and also used for the treatment of premenstrual dysphoric disorder.

Pharmaceutical form: Magrilan™ tablets were supplied as yellow/purple capsules containing an off-white powder. Each capsule contains the active ingredient, fluoxetine hydrochloride equivalent to 20 mg of fluoxetine, and inactive ingredients which include starch, gelatin, titanium dioxide and others.

Physical properties of active ingredient: Fluoxetine has a solubility of 14 mg/mL in water. It is a white crystalline solid with a MW 345.79.

Pharmacodynamics in vertebrates: Studies in animals suggest that fluoxetine is a potent uptake inhibitor of serotonin. Fluoxetine may bind to muscarinic, histaminergic and alpha1 adrenergic receptors and other membrane receptors. Its mechanism of action is linked to its inhibition of CNS neuronal uptake of serotonin.
(VI) A compound of formula 6:

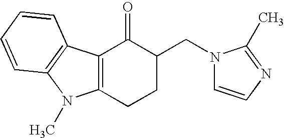

An example of a compound with this formula is ondansetron hydrochloride (ZFN) and a composition containing the compound is Zofran™.

Zofran™ (Glaxo Wellcome) contains the active ingredient ondansetron hydrochloride (ZFN)

Zofran™ tablets are indicated for the management of nausea and vomiting induced by cytotoxic chemotherapy and radiotherapy. they are also prescribed for the prevention and treatment of post-operative nausea and vomiting.

Pharmaceutical form: Zofran™ is supplied as yellow, oval film coated tablets containing 8 mg of the active ingredient ondansetron (as hydrochloride dihydrate), and containing the inactive ingredients lactose, microcrystalline cellulose, pregelatinized starch, hydroxypropyl methylcellulose, magnesium stearate, titanium dioxide and iron oxide yellow.

Physical properties of active ingredient: Ondansetron is a white powder, soluble in saline and water. It has a MW 365.9 (dihydrate form).

Pharmacodynamics in Vertebrates: Ondansetron is a selective 5-HT3 receptor antagonist. It is not a dopamine-receptor antagonist.
(VII) A compound of formula 7:

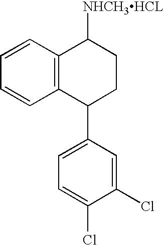

An example of a compound with this formula is sertraline hydrochloride (ZLT) and a composition containing the compound is Zoloft®.

Zoloft®(Pfizer), containing the active ingredient sertraline hydrochloride (ZLT)

Zoloft® is a prescription medicine used to treat depression, panic disorder, obsessive-compulsive disorder and post-traumatic stress disorder in adults.

Pharmaceutical form: Zoloft® is supplied as light blue film coated tablets containing the active ingredient Sertraline hydrochloride equivalent to 50 mg of sertraline, and the following inactive ingredients: dibasic calcium phosphate dihydrate, FD&C Blue #2 aluminium lake, hydroxypropyl cellulose, hydroxypropyl methylcellulose, magnesium stearate, microcrystalline cellulose, polyethylene glycol, polysorbate 80, sodium starch glycolate and titanium dioxide.

Physical properties of active ingredient: Sertraline hydrochloride is a white crystalline powder that is slightly soluble in water and isopropyl alcohol, and slightly soluble in ethanol. It has a MW 342.7.

Pharmacodynamics in vertebrates: Sertraline hydrochloride is a selective serotonin reuptake inhibitor (SSRI). The mechanism of action is linked to its inhibition of CNS neuronal uptake of serotonin (5HT).
(VIII) A compound of formula 8:

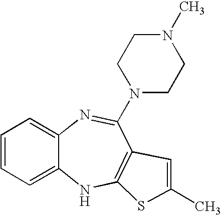

An example of a compound with this formula is olanzapine (ZPX) and a composition containing the compound is Zyprexa™.

Zyprexa™ (Lilly) contains the active ingredient, olanzapine (ZPX)

Zyprexa™ is prescribed for management of the manifestations of psychotic disorders.

Pharmaceutical form: Zyprexa™ is supplied as white gel coated tablets which is bright yellow inside. Each tablet contains 5 mg of the active ingredient, Olanzapine, and the inactive ingredients, carnuba wax, color mixture white, crospovidone, FD&C Blue #2 Aluminium lake, hydroxypropyl cellulose, hydroxypropyl methylcellulose, lactose, magnesium stearate, microcrystalline cellulose, titanium dioxide and indigo carmine colour (E132).

Physical properties of active ingredient: Olanzapine is a yellow crystalline solid, which is practically insoluble in water. It has a MW 312.44.

Pharmacodynamics in Vertebrates: Olanzapine is a psychotropic agent belonging to the thienobenzodiazepine class. It is a selective monoaminergic antagonist. The mechanism of action is unknown but activity may be mediated through a combination of dopamine and serotonin type 2 (5HT₂) antagonism, whilst side effects may be a result of olanzapine's antagonism of muscarinic M₁₋₅ receptors (anticholinergic), histamine H₁ and adrenergic alpha₁ receptors.

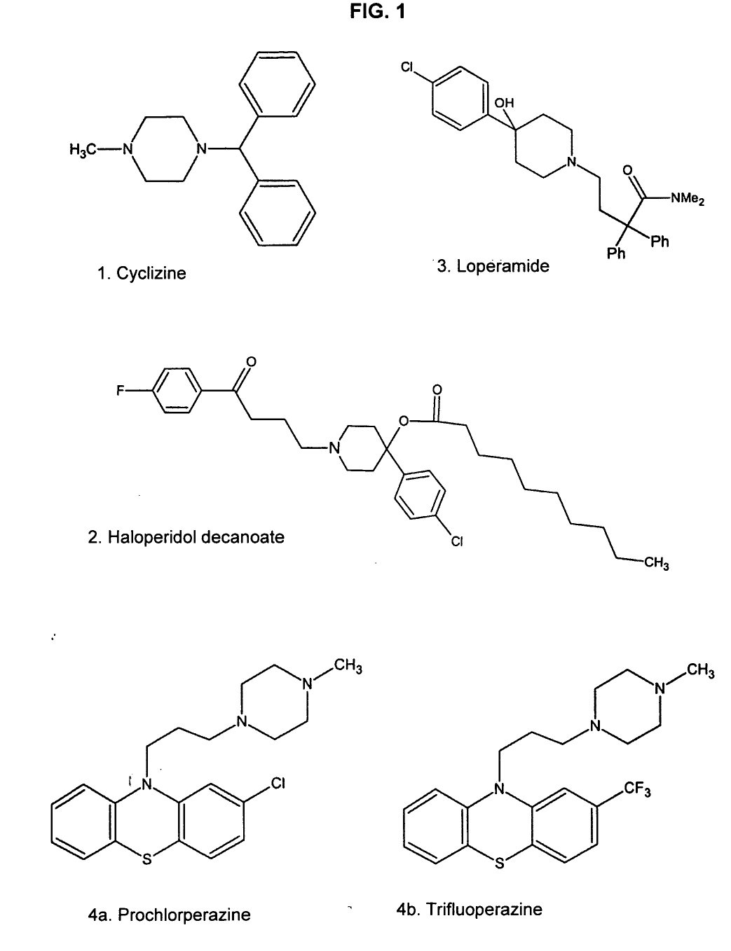

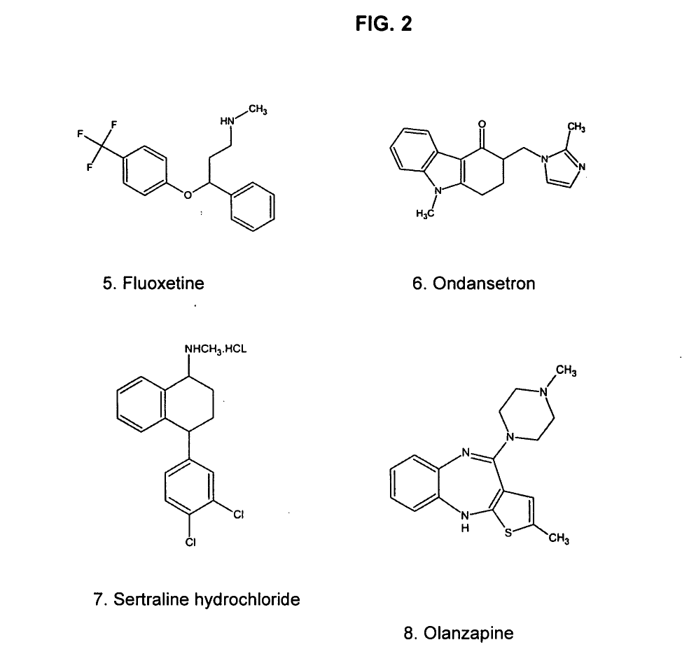

Table 1 provides a list of the pharmaceuticals covered in this application. In this section, basic information obtained from product information and published literature for the active ingredients is presented. The structures of the active ingredients are given in FIGS. 1 and 2.

TABLE 1
Pharmaceuticals used in the experiments

| | | | | Amount of | |
|---|---|---|---|---|---|
| | | | | active | Approx. |
| | | | | ingredient per | weight of |
| | | | | tablet as | whole |
| Drug Trade | | | | declared by | tablet |
| Name | REF | Manufacturer | Active Ingredient | manufacturer | (mg) |
| Marzine | CYZ | Pfizer | Cyclizine | 50 mg | 120 |
| Haldol | HDL | Pfizer | Haloperidol | 5 mg | 90 |
| | | | decanoate | | |
| Loperamil | IMD | DHA Pte Ltd | Loperamide | 2 mg | 90 |
| | | Singapore | hydrochloride | | |
| Magrilan | PZC | Mediochemie | Fluoxetine | 20 mg | 190 |
| | | Ltd | hydrochloride | | |
| Generic | PCZ | Malaysia | Prochlorperazine | 5 mg | 120 |
| | | Chemist Pte Ltd | maleate | | |
| Generic | TFZ | USP | Trifluoperazine | 5 mg | 130 |
| (analogue | | | hydrochloride | | |
| of PCZ) | | | | | |
| Zofran | ZFN | GlaxoWellcome | Ondansetron | 8 mg | 260 |
| | | | hydrochloride | | |
| Zyprexa | ZPX | Lilly | Olanzapine | 5 mg | 220 |
| Zoloft | ZLT | Pfizer | Sertraline | 50 mg | 160 |
| | | | hydrochloride | | |

A person skilled in the art will appreciate that the above compounds may be replaced by their corresponding salts within the scope and spirit of the present invention.

According to another aspect, the invention may also provide a compound for use as biocidal and/or biostatic or a composition comprising said compound, wherein the compound has a general formula R¹R²NR³, each of R¹, R² and R³ being defined as above, and wherein the compound is not at least one compound listed in Table 1. For example, there is also provided a compound of formula R¹R²NR³, wherein the compound is at least not CYZ. There is also provided a compound of formula R¹R²NR³, wherein the compound is at least not HDL. There is also provided a compound of formula R¹R²NR³, wherein the compound is at least not IMD. There is also provided a compound of formula R¹R²NR³, wherein the compound is at least not PZC. There is also provided a compound of formula R¹R²NR³, wherein the compound is at least not PCZ. There is also provided a compound of formula R¹R²NR³, wherein the compound is at least not TFZ. There is also provided a compound of formula R¹R²NR³, wherein the compound is at least not ZFN. There is also provided a compound of formula R¹R²NR³, wherein the compound is at least not ZPX. There is also provided A compound of formula R¹R²NR³, wherein the compound is at least not ZLT.

The composition according to the invention may comprise at least one compound of formula R¹R²NR³, according to any aspect of the invention, mixed to a binder. Any suitable binder for the purpose of the present invention may be used. In particular, the binder may be a curable binder. For example, a curable binder to form a mixture before applying the composition to the medium and then allowing the mixture to cure. The medium may be a solid medium, The medium may also be a liquid, for example an aqueous liquid. More in particular, water. The liquid may be contained in a container.

The binder according to the present invention may comprise at least one anti-fouling compound at less than standard concentration.

The compound(s) or composition(s) of the invention may be useful for biocidal and/or biostatic treatment. In particular, for the prevention of fouling and/or biofilm formation.

Accordingly, there is provided a method for biocidal and/or biostatic treatment of a medium comprising:
providing at least one compound having the formula R¹R²NR³, wherein R¹, R² and R³ are selected from the group consisting of hydrido, alkyl, aryl, heteroaryl, cycloalkyl, cycloaryl, cycloheteroaryl, alkenyl and alkynyl; and
applying the compound to the medium.

At least one compound is a substituted amine wherein R¹ and R² are selected from the group consisting of alkyl, heteroaryl, cycloalkyl, cycloaryl, cycloheteroalkyl, and cycloheteroaryl; and R³ is alkyl. R¹ and R² may form part of a carbocyclic or heterocyclic ring. In particular, R¹ and R² form part of a carbocyclic or heterocyclic ring, and wherein R³ is selected from the group consisting of methyl, ethyl, propyl and butyl.

More in particular, at least one compound is selected from the group consisting of: cyclizine, haloperidol decanoate, loperamide, prochlorperazine, trifluoperazine, fluoxetine, ondansetron, sertraline hydrochloride, olanzapine and salt(s) thereof.

The method may further comprise mixing the compound with a curable binder to form a mixture before applying the composition to the solid medium. In particular, the mixture is allowed to cure. In the method of the invention, the binder may comprise at least one anti-fouling compound at less than standard concentration. Preferably, the method is for the treatment is for prevention of fouling of the medium or for treatment is for prevention of biofilm formation.

There is also provide a coated substrate wherein the coating comprises at least one compound having the formula R¹R²NR³, wherein R¹, R² and R³ are selected from the group consisting of hydrido, alkyl, aryl, heteroaryl, cycloalkyl, cycloaryl, cycloheteroaryl, alkenyl and alkynyl.

The coated substrate may be any substrate which may be treated with any compound or composition according to the present invention. For example, the substrate may be any substrate exposed or in contact with a liquid, such as water. For example, a portion of or an entire vessel. The substrate may also be, for example, a container or portion thereof of standing water. The compounds or compositions according to the invention may also be used to treat re-circulated liquids, such as re-circulated water of fountains.

Having now generally described the invention, the same will be more readily understood through reference to the following examples which are provided by way of illustration, and are not intended to be limiting of the present invention.

### EXAMPLES

Effects on Marine Biofilm Bacteria

Growth Inhibition of Marine Bacteria by Non-Antibiotic Pharmaceuticals

Bacteria are very abundant in water, such as in the marine environment. Many can form biofilms on solid surfaces, which may be ship hulls or other submerged objects. Once formed, biofilms may modify the attachment behaviour of fouling macro-organisms such as barnacles, ship worms, etc (Maki et al., 1988; O'Connor, 1996; Maki et al., 2000; Huang and Hadfield, 2003). Microbial fouling involves the attachment of bacterial cells onto a surface, forming a biofilm. Following initial attachment of cells, multiple cell layers can be formed on top of this layer forming a biofilm. Organisms within biofilms are more resistant to antibiotics and cleaning agents.

For chemicals used in the environment, activity against bacteria has two implications. On one hand, bacterial activity is often responsible for breakdown of the antifouling agents in coatings, resulting in biodeterioration and poor performance. Microfouling bacteria are also a serious problem in fouling of membranes and heat exchanger surfaces. Novel antibacterial activity has important applications in water treatment systems. On the other hand, from an environmental perspective, persistent and strong antibacterial activity in chemical agents that are disposed into the marine environment can potentially result in impact on the natural micro-flora as well as development of more resistant strains of bacteria.

The object of the experiments herein below described was to examine the effect of these non-antibiotic pharmaceuticals listed in Table 1 against marine bacteria found in microfouling biofilms, using (1) disc inhibition to indicate whether the growth of bacteria is inhibited. This assay is a conventional method routinely used for screening antibacterial compounds to determine degree of susceptibility to antibacterial compounds. The diameter of the zone of inhibition is proportional to the degree of susceptibility of the bacterial strain. 9 drugs were tested against 26 strains of marine bacteria. As this method does not give information on-growth pattern such as the rate of growth or if inhibition was delayed, an additional (2) growth assay using 96-well plates was carried out. The rate of change in absorbance was measured. (3) In cases where inhibition occurred, there was a need to determine if the drugs were bacteriostatic or bacteriocidal to the bacteria so a count in the change in CFUs (=number of colony forming units) was done. (4) A bio-adhesion assay was also carried out to determine if the pharmaceuticals are able to modify bacterial attachment to hydrophilic or hydrophobic surfaces.

### Example 1

Isolation of Marine Bacteria from Singapore Marine Fouling Communities

Twenty six strains of marine bacteria were isolated from fouling communities located in the coastal waters around Singapore. They were collected by rubbing sterile swabs on the surfaces, then transferring the swabs to plates containing Marine Agar (Pronadisa #1802.00). Colonies were picked at random and then streaked repeatedly onto new agar plates to obtain pure cultures as determined by colony morphology. After isolation each strain of bacteria was frozen (−20° C.) in vials with glycerol (Ghema, 1994) from which new stock cultures were periodically established.

Bacterial motility was determined by observing swimming cells in broth cultures, and Gram stains were observed using light microscopy.

Results

Twenty six strains of marine bacteria were isolated from various surfaces in the marine environment near Singapore. The type of surface and its locations from which they were isolated, cell morphology, Gram staining and motility are presented in Table 2. They were representative of a range of morphologies, including rods and cocci, Gram positive and negative, and motile and non-motile strains. These bacteria isolated from the marine environment, were used in assessment of anti-bacterial activities of the pharmaceuticals.

TABLE 2
List of bacterial strains indicating collection source, response
to Gram staining, morphology and motility

| Strain | | Gram | | |
|---|---|---|---|---|
| Number | Source of Isolation | Strain | Morphology | Motility |
| 1 | PVC panel, Ponggol Marina | − | Rod | + |
| 2 | Polycarbonate panel, Ponggol Marina | − | Rod | 0 |
| 3 | Acrylic panel, Ponggol Marina | − | Rod | 0 |
| 4 | PVC Panel, Ponggol Marina | − | Rod | 0 |
| 6 | Plastic cable tie, Ponggol Marina | − | Short Rod | + |
| 7 | Oil slick on rocky shore, St John's Island | − | Rod | + |
| 8 | Under ascidian, Polyclinid ascidian on Siglap Buoy | − | Rod | + |
| 9 | Inside tube of Vermitid mollusc, Siglap Buoy | + | Rod | 0 |
| 10 | Inside sponge, Siglap Buoy | + | Rod | 0 |
| 11 | Under mixed fouling community, Siglap Buoy | − | Rod | 0 |
| 12 | On yellow encrusting organism, Siglap Buoy | + | Rod | 0 |
| 13 | Under sponge, Siglap Buoy | + | Rod | 0 |
| 14 | Under filamentous algae, Main Fairways Buoy | − | Rod | 0 |
| 16 | Under barnacle, Main Fairways Buoy | − | Rod | + |
| 17 | Under sponge, Main Fairways Buoy | − | Rod | 0 |
| 18 | Under bryozoan, Main Fairways Buoy | − | Rod | 0 |
| 19 | Under red encrusting sponge, Main | − | Coccus | 0 |
| | Fairways Buoy | | | |
| 20 | Under barnacle, St. John's Island | − | Coccus | 0 |
| 21 | Under barnacle, St. John's Island | − | Rod | + |
| 22 | Rock, St. John's Island | − | Rod | 0 |
| 24 | Rock, St. John's Island | + | Coccus | 0 |
| 26 | On zoanthid, St. John's Island | − | Coccus | 0 |
| 27 | On Pomatoleios krausii, St. John's Island | − | Rod | 0 |
| 28 | On plastic floats, Lim Chu Kang | + | Coccus | 0 |
| 29 | Under ascidian on rope, Changi fish farm | + | Rod | + |
| 30 | Under ascidian on rope, Changi fish farm | − | Rod | + |

### Example 2

Disk Inhibition Assay

Preparation of Drugs onto Disks for Assay

The non-antibiotic pharmaceuticals of Table 1 were prepared in the following way. A tablet with a known amount of active ingredient (as stated in the product information) was ground in a mortar and pestle and suspended in sterile deionized water to a concentration of 2 mg ml⁻¹. These samples were sonicated for at least 1 min. The exception was PZC, which was a capsule. The two halves of the capsule were separated and the powder released straight into a bottle and suitable amount of water added to make a concentration of 2 mg ml⁻¹. A small volume of each pharmaceutical solution or suspension was pipetted onto each 6 mm sterile disc (Macherey-Nagel #484000) to obtain either 25 μg or 50 μg concentration of active ingredient per disc. The drugs that do not dissolve in water form cakes on the disc and the caked side was flipped onto the agar surface.

As a comparison to the pharmaceuticals, to determine the susceptibility of the bacterial isolates to conventional antibiotics, commercially available antibiotic discs were used. Antibiotic discs (Oxoid® CT series) containing Ampicillin [Amp] (2 and 10 μg), Erythromycin [Ery] (5 and 15 μg), Streptomycin [Str] (10 and 25 μg) and Tetracycline [Tet] (5 and 30 μg) were placed on lawns of the bacterial strains inoculated as described below.

Assay Method

For all disk inhibition assays, bacterial cultures were grown in 5 ml of marine broth (Pronadisa #1217.00) in 15 ml centrifuge tubes on a rotary shaker, at 150 rpm, overnight. Sterile swabs dipped into the cultures were used to inoculate Marine Agar plates with a bacterial lawn by smearing the culture over the surface. After inoculating the plates, discs with antibiotics, pharmaceuticals or control blanks were placed on each plate. Each concentration of antibiotic or pharmaceutical was tested with three replicates on three separate occasions. After the disks were placed on the agar surface, plates were incubated overnight in the dark at 30° C. After incubation, plates were examined for zones of inhibition or clearing around control and treated disks. The diameter of any zone of clearing or inhibition was measured using Vernier calipers.

The assay was repeated three times with three replicates for each concentration used, each time. There was no zone of inhibition around the control blanks in all the assays.

Results

TABLE 3
Inhibition¹ of marine bacteria by selected antibiotics

| Bacteria | | | | | | | | |
|---|---|---|---|---|---|---|---|---|
| Strain/ | | | | | | | | |
| Antibiotic2 | | | | | | | | |
| and | | | | | | | | |
| Concentration | | | | | | | | |
| (μg) | Amp 2 | Amp 10 | Ery 5 | Ery 15 | Str 10 | Str 25 | Tet 5 | Tet 30 |
| 1 | ++ | +++ | − | − | ++ | +++ | − | − |
| 2 | − | ++ | − | +++ | − | − | − | − |
| 3 | − | − | ++ | +++ | − | − | − | − |
| 4 | +++++ | +++++ | +++++ | ++++ | ++ | ++ | ++ | ++ |
| 6 | − | − | − | ++ | − | ++ | − | + |
| 7 | + | ++ | ++ | ++ | ++ | ++ | − | + |
| 8 | +++ | ++++ | +++ | ++++ | ++ | ++ | + | ++ |
| 9 | ++ | ++++ | ++++ | ++++ | ++ | ++ | ++ | ++ |
| 10 | ++++ | +++++ | ++++ | +++++ | ++ | ++ | ++++ | ++ |
| 11 | +++ | ++++ | +++ | +++ | ++ | +++ | − | − |
| 12 | − | − | − | ++ | − | ++ | − | ++++ |
| 13 | ++ | ++ | +++ | +++ | ++ | ++ | + | ++ |
| 14 | ++ | +++ | − | ++ | − | + | − | ++ |
| 16 | + | ++ | ++ | + | + | ++ | − | − |
| 17 | − | − | +++++ | +++++ | ++ | ++ | − | − |
| 18 | +++ | +++ | + | ++ | ++ | ++ | − | + |
| 19 | +++ | +++ | +++ | +++ | − | − | − | − |
| 20 | − | +++ | ++ | +++ | − | − | − | ++ |
| 21 | − | − | +++ | +++ | ++ | ++ | − | ++ |
| 22 | ++ | ++ | ++ | ++++ | + | +++ | + | ++ |
| 24 | ++++ | +++++ | +++ | +++ | − | +++ | − | − |
| 26 | ++ | ++ | +++ | +++ | − | +++ | − | − |
| 27 | +++ | ++++ | +++ | +++ | − | − | − | ++ |
| 28 | + | ++ | +++ | +++ | ++ | ++ | − | ++ |
| 29 | ++++ | ++++ | ++++ | +++ | ++ | ++ | ++ | +++ |
| 30 | ++++ | +++ | +++ | +++ | ++ | ++ | ++ | ++ |

¹For zone of inhibition: − = diameter <6 mm; + = diameter 6-9 mm; ++ = diameter 10-19 mm; +++ = diameter 20-29 mm; ++++ = diameter 30-39 mm; +++++ = diameter >40 mm.
²Antibiotics: Amp = ampicillin; Ery = erythromycin; Str = streptomycin; Tet = tetracycline.

The results from the assays (Table 3) using the commercial antibiotic discs indicated that all the isolated strains were susceptible to at least one antibiotic. Strain 3 was the least susceptible, being only inhibited by Ery. Twelve of the isolates (4, 7, 8, 9, 10, 13, 14, 18, 22, 28, 29, 30) were susceptible to all four antibiotics. Of the four antibiotics, Tet was very effective against strain 12 at 30 μg but otherwise was the least effective against most strains of marine bacteria. The inhibition of growth was not always proportional to the concentration of the antibiotics. Greater inhibition at lower concentrations of antibiotics was seen for strains 4 and 29, against Ery at 5 μg.

The strongest activity of the non-antibiotic pharmaceuticals observed was for PZC, PCZ, ZLT, and TFZ (Table 4). All of these pharmaceuticals were effective against both Gram positive and negative bacteria.

TABLE 4
Inhibition¹ of marine bacteria by ten pharmaceuticals sorted
according to the strains most inhibited by all four drugs

| Bacterial | | | | | | | | |
|---|---|---|---|---|---|---|---|---|
| Strain/ | | | | | | | | |
| Compound2 | | | | | | | | |
| and Con- | | | | | | | | |
| centration | | ZLT | PZC | PZC | TFZ | TFZ | PCZ | PCZ |
| (μg) | ZLT 25 | 50 | 25 | 50 | 25 | 50 | 25 | 50 |
| 8 | ++ | ++ | ++ | +++ | + | ++ | ++ | ++ |
| 20 | +++ | +++ | ++ | ++ | ++ | +++ | ++ | ++ |
| 12 | ++ | +++ | ++ | +++ | ++ | ++ | ++ | +++ |
| 17 | ++ | ++ | ++ | +++ | + | ++ | ++ | +++ |
| 19 | +++ | ++++ | ++ | +++ | ++ | ++ | ++ | ++ |
| 3 | +++ | +++ | ++ | ++ | ++ | ++ | ++ | ++ |
| 18 | ++ | ++ | ++ | ++ | ++ | ++ | ++ | +++ |
| 29 | ++ | ++ | ++ | +++ | ++ | ++ | ++ | ++ |
| 30 | ++ | +++ | ++ | ++ | ++ | ++ | ++ | ++ |
| 4 | ++ | ++ | ++ | +++ | ++ | ++ | ++ | ++ |
| 9 | ++ | ++ | ++ | ++ | ++ | ++ | ++ | ++ |
| 22 | +++ | +++ | ++ | ++ | ++ | +++ | ++ | ++ |
| 27 | ++ | ++ | +++ | +++ | ++ | ++ | + | ++ |
| 10 | ++ | ++ | ++ | ++ | ++ | ++ | ++ | ++ |
| 11 | ++ | ++ | ++ | +++ | ++ | ++ | ++ | ++ |
| 13 | ++ | ++ | ++ | ++ | ++ | ++ | ++ | ++ |
| 24 | ++ | ++ | + | ++ | ++ | ++ | ++ | ++ |
| 28 | ++ | ++ | ++ | ++ | + | ++ | ++ | ++ |
| 26 | ++ | ++ | + | + | + | ++ | + | ++ |
| 21 | ++ | ++ | + | ++ | + | + | − | ++ |
| 2 | ++ | ++ | ++ | ++ | − | ++ | − | − |
| 16 | + | + | + | ++ | + | ++ | − | − |
| 6 | + | ++ | − | − | + | + | − | + |
| 14 | − | +/− | ++ | ++ | − | − | − | − |
| 7 | − | + | − | ++ | − | + | − | − |
| 1 | +/− | + | − | + | − | − | − | − |

¹For zone of inhibition: +/− = at least one treatment resulted in a zone of inhibition >5 mm diameter; + = diameter 5-9 mm; ++ = diameter 10-19 mm; +++ = diameter 20-29 mm; ++++ = diameter 30-39 mm. (−) = denotes no zone of inhibition >5 mm diameter was observed for all treatments.

PZC at a concentration 25 μg inhibited 23 out of 26 strains and only strain 6 was not inhibited at 50 μg. TFZ at 25 μg concentration inhibited 22 out of 26 strains of bacteria, and only 2 strains showed no inhibition at 50 μg. ZLT was inhibiting to all but 2 strains at 25 μg while inhibitory to all at 50 μg.

ZLT at 25 μg had slight inhibition on strain 1 while PZC at the same concentration inhibited the growth of strain 14 but all drugs had no effect on strain 7 at the same concentration.

Those strains, 2, 3, 19, 20, 27 that were not inhibited by 25 μg of Str were inhibited by ZLT, PZC, PCZ and TFZ at 25 μg (except strain 2 which was not inhibited by 25 μg of TFZ and PCZ).

The size of the zone of inhibition for strains affected by the pharmaceuticals, even at 50 μg of drugs was equal to or less than the zone of inhibition of Amp and Ery at lower concentration.

Strains 4, 8, 9, 10, 13, 22, 28, 29 and 30 that showed inhibition to all concentrations of antibiotics were also inhibited by the more potent drugs like ZLT, PZC, PCZ and TFZ. However, the converse was not true. Strains 3, 12, 17, 20, 21, although inhibited by all the drugs were resistant to some of the antibiotics.

### Example 3

Growth Assay

To prevent possible contamination from bacteria found on the tablets, the drugs were sterilised by putting the ground up pills powders into a small volume of absolute alcohol for half an hour. The alcohol was then removed by blow-drying with air passed through a sterile 0.2 um (Sartorius #16534) syringe filter. The dried powder was re-suspended in sterile deionized water and made up to 2 mg ml⁻¹.

The same bacterial strains were used and cultured the same way as in disc inhibition assay. Cells were harvested by centrifugation at 300 rpm for 10 min. The supernatant was discarded and the pellet re-suspended using sterile seawater collected from St. John's island, Singapore. The concentration of bacterial cells was adjusted to an absorbance of approximately 0.6 using a spectrophotometer (Beckman DU530) at wavelength 600 nm.

The growth with response to the pharmaceuticals was assumed to be proportional to the absorbance of the bacteria. Growth of the bacteria was studied with fixed final concentration of 25 μg ml⁻¹ of four of the more inhibitory pharmaceuticals in the disk assay (PZC, PCZ, ZLT and TFZ) in flat bottomed 96-well tissue culture plates (Nunclon™ #167008). To each well 25 μl of each drug solution (concentration adjusted to 100 μg ml⁻¹) were pipetted into three wells followed by 10 μl of broth and 65 μl of sterile seawater. For wells that were to contain bacteria, 60 μl of sterile seawater was used instead. All wells used for growth studies were prepared in triplicate and inoculated with 5 μl of the bacterial suspension. Blanks consisting of the respective volumes of pharmaceutical with Marine Broth and distilled water, if needed, were prepared in triplicate and inoculated with 5 μl of sterile seawater.

The absorbance (A₆₀₀) of each well was read in a plate reader (Sunrise Tecan Model F039300) immediately (time=0 hours) and after 1, 2, 3, 4, 20, 23 and 28 hours. Plates were incubated overnight inside a moistened airtight container.

Any changes in absorbance were plotted and compared with control. The mean absorbance (mean bac) from the bacteria was obtained by deducting the absorbance values of blank wells (containing seawater and broth only) from that for the control wells (containing bacteria in seawater and broth only). The absorbance arising from the drugs was obtained from the 0 hr reading for wells containing drugs and bacteria, minus the wells with drugs alone (mean “drug”). The error bars given in the figures are standard error. The growth assays were repeated three times.

Results

The growth responses of all strains of bacteria at 25 μg ml⁻¹ of pharmaceuticals are presented in Table 5.

TABLE 5
Inhibition of bacterial growth by 25 μg ml⁻¹ of pharmaceuticals

| Strain | ZLT | PZC | TFZ | PCZ |
|---|---|---|---|---|
| 1 | + | 0 | 0 | 0 |
| 2 | +++ | + | 0 | 0 |
| 3 | +++ | + | +++ | +++ |
| 4 | +++ | 0 | +++ | +++ |
| 6 | + | 0 | + | 0 |
| 7 | 0 | + | + | + |
| 8 | +++ | + | + | +++ |
| 9 | +++ | + | +++ | +++ |
| 10 | +++ | +++ | +++ | +++ |
| 11 | + | + | + | + |
| 12 | +++ | + | +++ | +++ |
| 13 | +++ | + | +++ | +++ |
| 14 | 0 | 0 | 0 | 0 |
| 16 | + | 0 | 0 | 0 |
| 17 | +++ | +++ | +++ | +++ |
| 18 | +++ | + | +++ | +++ |
| 19 | +++ | + | +++ | +++ |
| 20 | +++ | + | + | + |
| 21 | + | 0 | 0 | 0 |
| 24 | +++ | 0 | + | 0 |
| 26 | + | 0 | 0 | 0 |
| 27 | +++ | + | +++ | + |
| 28 | +++ | 0 | +++ | 0 |
| 29 | +++ | + | +++ | + |
| 30 | +++ | +++ | +++ | +++ |

0 denotes no inhibition on growth, p is not significant;
+++ denotes little or no growth, p < 0.001;
+ denotes growth present but inhibited, p < 0.01

TABLE 6
Inhibition of bacterial growth by 50 μg ml⁻¹ of pharmaceuticals

| Strain | ZLT | PZC | TFZ | PCZ |
|---|---|---|---|---|
| 1 | +++ | 0 | 0 | 0 |
| 4 | +++ | +++ | +++ | +++ |
| 9 | +++ | +++ | +++ | +++ |
| 14 | 0 | 0 | 0 | 0 |
| 18 | +++ | +++ | +++ | +++ |
| 20 | +++ | +++ | +++ | +++ |
| 27 | +++ | +++ | +++ | +++ |
| 28 | +++ | +++ | +++ | +++ |
| 29 | +++ | +++ | +++ | +++ |
| 30 | +++ | +++ | +++ | +++ |

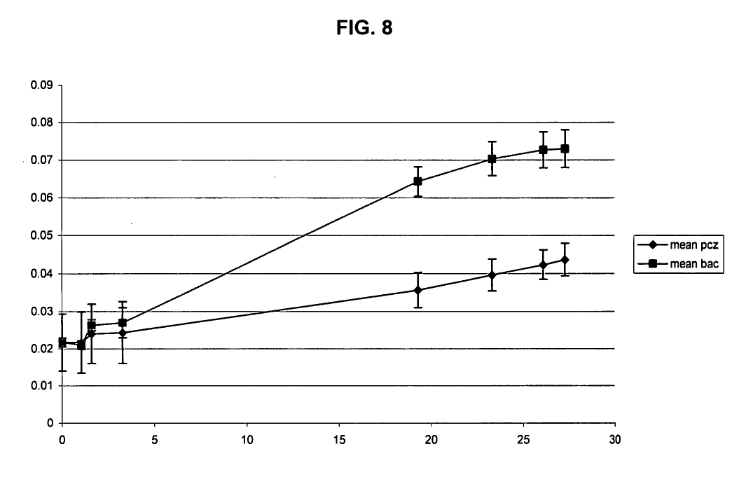

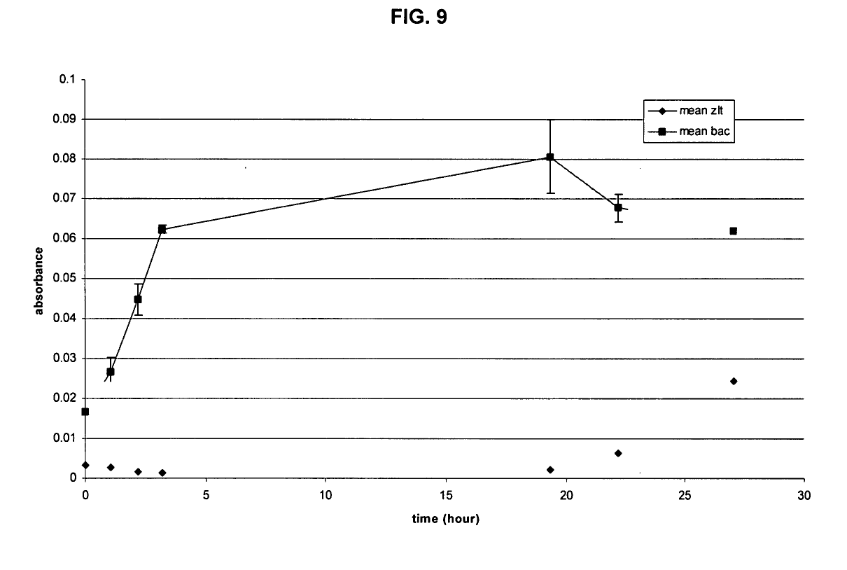

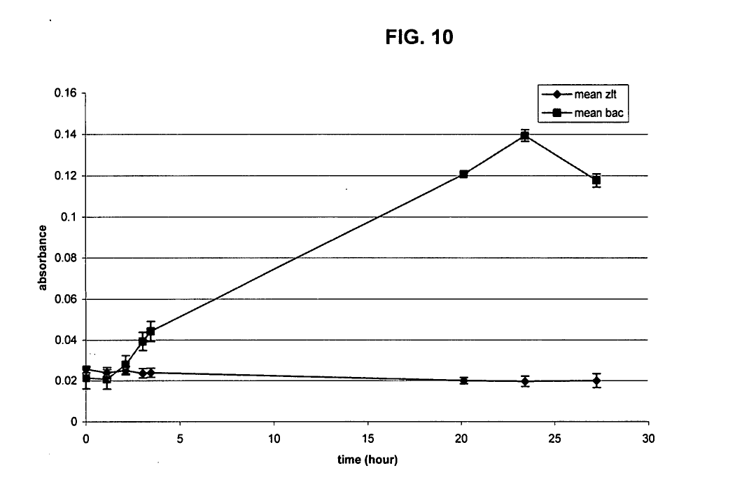

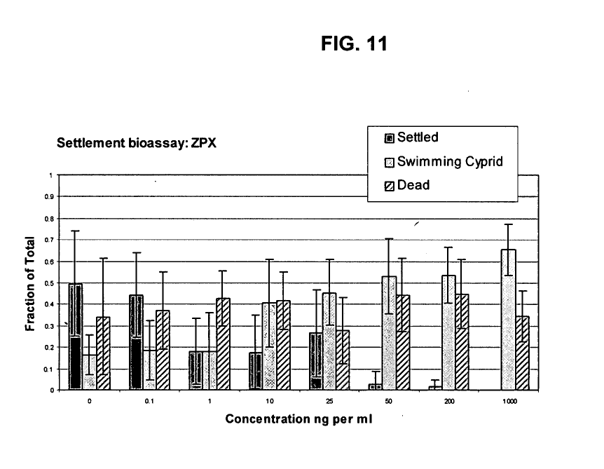

The control treatments showed standard log phase growth. When inhibited, the growth was reduced resulting in lowered absorbance value (see FIG. 8). When growth was absent, the absorbance remained the same and when there was cell lysis, the absorbance decreased (FIG. 10). As seen in Table 5, PZC strongly inhibited growth on 3 strains of bacteria, PCZ acted similarly on 11 strains, TFZ on 14 strains and 17 strains by ZLT. Strain 16 showed delayed growth after 20 hours in the presence of ZLT (FIG. 9).

Results of the 24 hours growth assay at 25 μgml⁻¹ of pharmaceuticals were similar to the disc inhibition except for strains 4, 14, 16, 21, 24, 26 and 28. The drugs responsible for the discrepancies were PCZ (3 strains), PZC (7 strains) and TFZ (3 strains). Growth in these strains was not inhibited whereas there was inhibition in disc assay.

### Example 4

Bacteriostatic vs Bacteriocidal Activity from Growth Assay: CFU Counts

Growth of the bacteria was re-examined for four drugs (PZC, PCZ, TFZ and ZLT) that exhibited inhibitory action in the disk and growth assays, at the high concentration of 50 μg ml⁻¹. Bacteria strains (1, 4, 9, 14, 18, 20, 27, 28, 29, 30) representing a variety of morphological types were used.

The assay was carried out using flat bottomed 96 well tissue culture plates (Nunclon™ #167008). For each well, 50 pt of a 100 μg ml⁻¹ drug solution in sterile distilled water, was added to 45 μl of Marine Broth. Controls for growth consisted of 50 μl of sterile deionized water and 45 μl of marine broth. All wells used for growth studies were prepared in triplicate and inoculated with 5 μl of the bacterial suspension. Blanks consisting of the above volumes of pharmaceutical with Marine Broth and distilled water, were prepared in triplicate and inoculated with 5 μl of sterile seawater. Immediately after adding all the components to the wells for the growth studies, 10 μl was removed and used to inoculate plates of Marine Agar to determine colony-forming units (CFU). The absorbance (A₆₀₀) of each well was read in the plate reader immediately.

The inoculated plates of Marine Agar were incubated overnight on a rotary shaker (150 rpm) at 30° C. inside a moistened airtight container. After incubation, the absorbance was read again and another 10 μl was removed and used to inoculate Marine Agar plates to determine CFU.

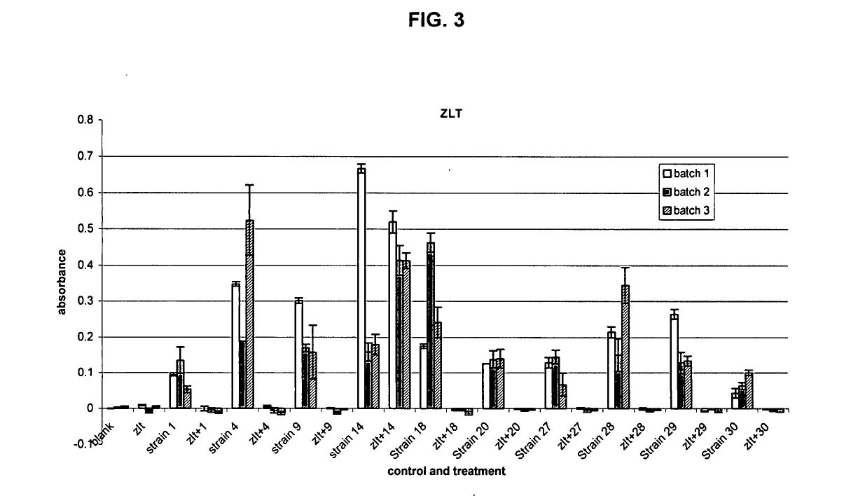

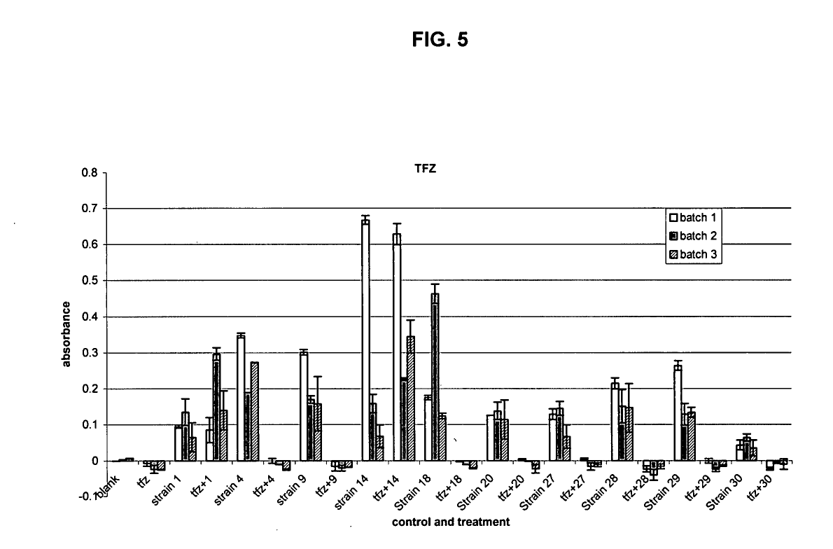

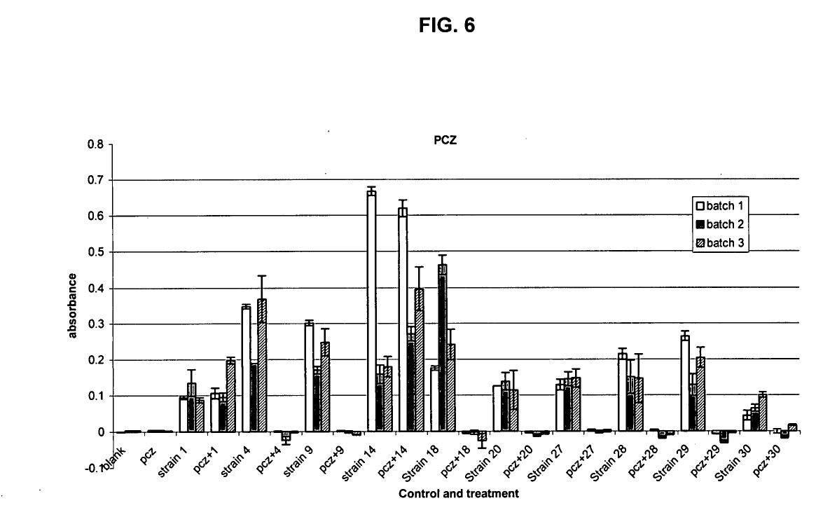

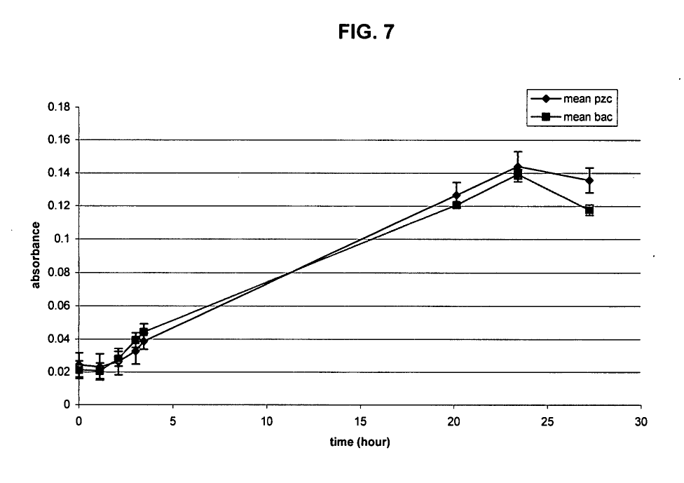

The growth responses of all strains after 24 hours for 25 μg ml⁻¹ and 50 μg ml⁻¹ were summarized in Table 5 and 6 respectively. The difference in absorbance over 24 hours for each bacterial strain versus controls and treatments were plotted in FIGS. 3 to 6 for each of the four drugs. The data was analyzed using 1-tailed t-test. The mean values of CFU counts were presented in Table 7.

TABLE 7
Colony forming units for ten bacterial strains in response to four drugs

| Strain | PZC Result | | PCZ Result | | ZLT Result | | TFZ Result | |
|---|---|---|---|---|---|---|---|---|
| 1 | 0 | no effect | 0 | no effect | ↓ | bacteriostatic | 0 | no effect |
| 4 | ↓ | bacteriostatic | x | bacteriocidal | x | bacteriocidal | x | bacteriocidal |
| 9 | ↓ | bacteriostatic | x | bacteriocidal | x | bacteriocidal | x | bacteriocidal |
| 14 | 0 | no effect | 0 | no effect | ↑ | no effect | 0 | no effect |
| 18 | ↓ | bacteriostatic | ↓ | bacteriostatic | ↓ | bacteriostatic | ↓ | bacteriostatic |
| 20 | ↓ | bacteriostatic | ↓ | bacteriostatic | x | bacteriocidal | ↓ | bacteriostatic |
| 27 | x | bacteriocidal | x | bacteriocidal | x | bacteriocidal | x | bacteriocidal |
| 28 | ↓ | bacteriostatic | ↓ | bacteriostatic | ↓ | bacteriostatic | ↓ | bacteriostatic |
| 29 | ↓ | bacteriostatic | x | bacteriocidal | x | bacteriocidal | x | bacteriocidal |
| 30 | ↓ | bacteriostatic | ↓ | bacteriostatic | ↓ | bacteriostatic | ↓ | bacteriostatic |

0: No change in CFU count after 24 hours
↑: Increase in CFU count after 24 hours
↓: Decrease in CFU count after 24 hours
X: No growth on plates

Results

Strain 1 was killed by 50 μgml⁻¹ of ZLT only while strain 14 was not killed by 50 μgml−1 concentrations of the PZC, PCZ, ZLT and TFZ. Those bacteria that were not killed showed that the drugs only had bacteriostatic effect. The four drugs at 50 μgml⁻¹ were bacteriocidal against all the other eight strains. Bacteriocidal effect is reflected by decreased in absorbance and the CFU counts. In many instances, the CFU counts were zero or close to it (Table 7).

### Example 5

Bio-Adhesion Assay

This assay identifies the preference of each bacterial strain, for either hydrophilic or hydrophobic surfaces, and determines if the pharmaceuticals are able to modify bacterial attachment to these two surfaces. Hydrophobic interactions are, in many instances, responsible for microbial adhesion. Agents that inhibit adhesion by interfering with hydrophobic effect have been studied in polystyrene were hydrophobic salivary components (Eli et al., 1989), Tween 20 (Klotz et al., 1985), various graft copolymers, (Humphries et al., 1987), caseinoglycopeptides (Neeser et al, 1988) and lipopolysaccharides (Pringle, 1988).

Materials and Methods

The effect of pharmaceuticals on bacterial adhesion was analyzed using a modified microplate method adapted from Shea and Williamson (1990). This method consists of staining the attached bacteria with crystal violet, releasing the stain with sodium desoxycholate, and measuring the resulting absorbance of the crystal violet. The absorbance is proportional to the degree of bacterial adhesion to the surface. Shea and Williamson (1990) demonstrated a positive correlation between the CFUs and resulting absorbance.

Ten strains of bacteria (strain numbers 1, 4, 9, 14, 18, 20, 27, 28, 29 and 30) were chosen for this study. Flat bottom non-tissue culture treated polystyrene (hydrophobic, Falcon #351172) (referred to as polystyrene) and flat bottom tissue culture treated polystyrene (hydrophilic, Falcon #353072) microplates were used in the analyses. These two types of plates were chosen because they were sterile and offered two different kinds of surfaces to determine the adhesion preferences of the bacteria. Each test was done in duplicate. For the control and blank wells, 50 μl of sterile deionized water were added. For those wells that were to contain drugs, 50 μl of drugs at concentration of 125 μg ml⁻¹ were added. Then 200 μl of bacteria suspension in sterile seawater, whose absorbance had been adjusted to 0.6 at 600 nm using a spectrophotometer (Beckman), were added. Blanks contained 200 μl of sterile seawater.

The bacteria in suspension were allowed to attach for 2 hours at 28° C. Unattached cells were removed by pipetting out almost all trace of liquid and rinsed three times with 300 μl of sterile seawater each time. The attached cells were stained with 200 μl of crystal violet solution (0.1% w/v) in each well for 5 min. After removing the stain by pipetting, the wells were rinsed twice with 300 μl of sterile deionized water each time. Sodium desoxycholate (200 μl) solution (2% w/v) was then added to each well to extract the crystal violet from the stained cells for 10 min. The absorbance of the extracted solution was read at 590 nm using a microplate reader (Sunrise, Tecan, Switzerland).

The data were analyzed using T-test (Two-sample assuming unequal variances) to determine the significance of the absorbance values of each bacterial strain versus the blanks to determine whether there was adhesion, i.e. increase in absorbance. Each drug effect on adhesion was analyzed by comparing the absorbance of each bacterial strain minus the control blank versus bacteria in presence of drug minus the drug control. When the data is significant, there are bacterial cells attached to the surface in the control but in the presence of drugs, there is no attachment.

Results

The adhesion of the different bacteria to the two types of surfaces is presented in Table 8, and the effect with pharmaceutical treatment is given in Table 9.

TABLE 8
Adhesion of bacteria to tissue culture treated polystyrene (hydrophilic
surface) and untreated polystyrene surfaces (hydrophobic surface)

| Bacteria | | |
|---|---|---|
| Strain No | Hydrophilic | Hydrophobic |
| 1 | NS | * |
| 4 | ** | *** |
| 9 | * | *** |
| 14 | *** | *** |
| 18 | NS | * |
| 20 | * | *** |
| 27 | * | *** |
| 28 | ** | *** |
| 29 | * | *** |
| 30 | *** | * |

Significantly more adhesion to surface than in control:
*** p < 0.001,
** p < 0.005,
* p < 0.05,

TABLE 9
Effect of drugs on adhesion of bacteria

| | Strain | Hydrophilic | Hydrophobic |
|---|---|---|---|
| Drug | Number | surface | surface |
| CYZ | 1 | NS | NS |
| | 4 | NS | NS |
| | 9 | NS | NS |
| | 14 | NS | NS |
| | 18 | NS | NS |
| | 20 | NS | NS |
| | 27 | *Incr adhesion | NS |
| | 28 | NS | NS |
| | 29 | NS | NS |
| | 30 | NS | NS |
| HDL | 1 | NS | *** |
| | 4 | NS | NS |
| | 9 | NS | NS |
| | 14 | NS | NS |
| | 18 | NS | NS |
| | 20 | NS | * |
| | 27 | NS | NS |
| | 28 | NS | NS |
| | 29 | NS | NS |
| | 30 | NS | NS |
| IMD | 1 | *incr adhesion | * |
| | 4 | NS | NS |
| | 9 | NS | NS |
| | 14 | * | NS |
| | 18 | NS | NS |
| | 20 | NS | NS |
| | 27 | NS | NS |
| | 28 | NS | NS |
| | 29 | NS | NS |
| | 30 | NS | NS |
| PZC | 1 | NS | * |
| | 4 | NS | NS |
| | 9 | NS | * |
| | 14 | NS | NS |
| | 18 | NS | NS |
| | 20 | NS | NS |
| | 27 | NS | NS |
| | 28 | NS | NS |
| | 29 | NS | NS |
| | 30 | NS | NS |
| PCZ | 1 | NS | * |
| | 4 | * | * |
| | 9 | NS | * |
| | 14 | NS | NS |
| | 18 | NS | NS |
| | 20 | NS | NS |
| | 27 | NS | NS |
| | 28 | NS | NS |
| | 29 | NS | NS |
| | 30 | NS | * |
| TFZ | 1 | NS | NS |
| | 4 | * | *** |
| | 9 | NS | ** |
| | 14 | NS | NS |
| | 18 | * | NS |
| | 20 | NS | NS |
| | 27 | NS | *** |
| | 28 | NS | NS |
| | 29 | NS | NS |
| | 30 | NS | NS |
| ZFN | 1 | NS | NS |
| | 4 | NS | ** |
| | 9 | NS | *** |
| | 14 | NS | * |
| | 18 | NS | * |
| | 20 | NS | ** |
| | 27 | NS | *** |
| | 28 | NS | *** |
| | 29 | NS | * |
| | 30 | * | * |
| ZLT | 1 | NS | *** |
| | 4 | NS | *** |
| | 9 | NS | * |
| | 14 | NS | NS |
| | 18 | NS | NS |
| | 20 | NS | *** |
| | 27 | NS | *** |
| | 28 | NS | *** |
| | 29 | NS | NS |
| | 30 | *** | NS |
| ZPX | 1 | NS | *** |
| | 4 | NS | * |
| | 9 | NS | *** |
| | 14 | NS | ** |
| | 18 | NS | * |
| | 20 | NS | *** |
| | 27 | NS | *** |
| | 28 | NS | *** |
| | 29 | NS | * |
| | 30 | * | NS |

Significantly less adhesion observed:
* p < 0.05
** p < 0.01,
*** p < 0.005

All the bacteria strains except strain 30, showed greater adhesion to polystyrene surface. Strain 14 was adhesive to both surfaces. Strain 1 and 18 did not adhere to tissue culture surface.

Where there were negative mean values, the blanks showed greater absorbance than when bacteria were present. This was because the drugs themselves were not soluble and some drugs may have remained in the wells due to pipetting error and interacted with the dye thus increased the absorbance. However, such instances were not common.

The significant values displayed by the bacteria and the polystyrene surfaces are indicators of strong cellular adhesion. Where there was strong adhesion, the drugs' effects in blocking adhesion become apparent. The low significance in the tissue culture dishes showed a lack of preference by the bacteria to this surface. The effect of low bacterial adhesion resulted in equally low effects from the drugs.

The majority of bacterial isolates in this study attached in greater numbers to the hydrophobic substratum, polystyrene, than to the hydrophilic tissue culture plates. The exceptions were strain 14, which attached equally well to both substrata, and strain 30 which attached in greater numbers to the hydrophilic substratum. The inhibitory effect by the drugs on the bacteria was only evident when the adhesion to the substrata was strong.

Some of the drugs increased adhesion to polystyrene: Strain 4 in TFZ, strain 27 in CYZ and strain 1 in IMD and PCZ, and strain 30 in ZFN.

In the previous growth assay (Table 5), ZLT inhibited growth of all strains of bacteria except strain 14. In the adhesion assay (Table 9), at least some cells of strain 14 and 29 remained attached to the polystyrene surface in the presence of ZLT. Also, TFZ inhibited growth on strains 4, 9 and 27 (Table 5) and there was significant inhibition of adhesion observed in this assay. PZC inhibited growth and adhesion of strain 9. PCZ significantly inhibited growth of 4, 9 and 30, and also significantly prevented cell adhesion in these strains.

The presence of CYZ did not modify bacterial adhesion on polystyrene but it did marginally increased adhesion for strain 27 in the tissue culture surface. For other drugs in polystyrene surface, HDL caused significant inhibition of adhesion in strain 1 and 20. IMD caused greater adhesion in strain 1, PZC on strains 1 and 9 and PCZ on strains 1, 4, 9 and 30. IMD also caused strain 1 to adhere to tissue culture surfaces.

ZPX and ZFN prevented biofilm formation for almost all strains in the polystyrene surface. However ZFN increased cell adhesion in the tissue culture surface for strain 30.

Discussion (for Examples 2-5)

The compounds CYZ, IMD, ZPX, ZFN and HDL did not have any appreciable antibiotic activity against marine bacteria, although ZPX and ZFN prevented normal adhesion of bacteria to polystyrene.

This study has found some bacterial strains that were resistant or sensitive to only high concentrations of four standard antibiotics, yet other strains that were sensitive to all four antibiotics. Ery was effective against most of the strains of marine bacteria, followed by Amp, Str and Tet. The inhibitory responses, i.e. size of the zone of inhibition, to antibiotics when they were strongest were larger than that of any of the pharmaceuticals. Drug concentration at 25 μgml−1 was sufficient to inhibit many strains of bacterial growth. When this concentration didn't inhibit, doubling it was effective, except for strain 1 and 14.

However, those strains that were resistant to two or more conventional antibiotics were inhibited by four of the pharmaceuticals, ZLT, PZC and PCZ and TFZ. These drugs may have promise as anti-fouling compounds. ZLT in particular stood out as a wide spectrum anti-microbial compound. However, the mode of action on the bacteria is unknown.

The mechanism of action on vertebrates is similar for PZC, HDL, ZPX and ZFN as they are all Type 5 HT inhibitors. However, only PZC had inhibitory activity on bacterial growth. Antibacterial activity has been noted for TFZ. It is postulated that since TFZ is a calmodulin antagonist, it could be acting on proteins, which normally bind to calcium thus making the protein inactive, thereby affecting calcium driven metabolic pathways. PZC and ZLT are both anti-depressants. Currently we do not know the exact mechanism of their inhibitory action on bacterial growth.

The four drugs PZC, PCZ, TFZ and ZLT not only inhibited growth in some of the strains of bacteria but also prevented cell adhesion. There appears to be some relationship between inhibiting of cell growth and preventing adhesion. However if the drugs were slow acting, the bacteria could go into biofilm mode and so are no longer in direct contact with the drugs thus ensuring their survival.

ZPX, ZFN showed the most promise as anti-adhesion compounds on hydrophobic surfaces. Since these two drugs had no effect on growth inhibition, there may not be strong selective pressure to overcome these drugs. These drugs are novel as instead of conventional antibiotic action that affects bacterial replication, they appear to prevent the bacterium from attaching. Pharmaceuticals that don't encourage adhesion have potential in prosthetics anti-adhesion therapy and in anti-fouling. Once bacteria form a biofilm, they are more difficult to be removed by conventional antibiotics.

### Example 6

Identification of Bacteria Isolates Used for Screening for Anti-Biofilm Activity

Morphological examination and Gram stains were performed at least three separate times using Gram stain control slides (MicroBioLogics, St. Cloud, Minn.) with Escherichia coli and Staphylococcus aureus as the negative and positive controls, respectively. Motility was determined by microscopic examination of broth cultures for swimming cells.

An identification system for non-fastidious, non-enteric Gram negative rods api® 20 NE (Biomérieux, France) was used to identify the Gram negative rods to the level of genus according to the manufacturer's instructions.

Methods Used for Identification of Bacteria by 16S rDNA

Initial identification of cultures was accomplished by sequencing of the 16S rDNA gene after amplification with the polymerase chain reaction (PCR). The 16S rDNA was amplified either directly from cells or from DNA extracted from the cells using Bacterial domain specific primers (27F, 5′ AGAGTTGATCATGGCTCAG 3′ (hereinafter also referred to as SEQ ID NO: 1) and 1492R, 5′ TACGGYTACCTTGTTACGACTT 3′ (hereinafter also referred to as SEQ ID NO: 2); Lane 1991). PCR amplifications were performed using a Perkin Elmer Gene Amp PCR System 9600 thermal cycler. Controls without DNA or cells were used in all sets of PCR reactions to ensure that there was no contaminating template. PCR products were examined on 1% agarose gels by staining with ethidium bromide. To confirm their identity, PCR products were sequenced using an ABI Prism 377 DNA Sequencer (Applied Biosystems) at 1^{st} Base Pte Ltd, Singapore. Resulting sequences were used to identify bacterial isolates to the family or genus level with BLAST (Altschul et al 1997). In addition, a phylogenetic tree was constructed using the neighbour-joining algorithm with a Juke-Cantor parameter in the MEGA program (bootstrapping number 1000), to determine the phylogenetic affinities. Methanococcus vannielii (M36507) was selected as out-group.

Results of 16S rDNA Analysis

The results are given in Table 10. From the behaviour of the bacteria in the bioassays, as well as colony characteristics, we continue to recognise these as 24 different strains.

TABLE 10
Bacteria isolated from different locations in Singapore waters and used in the assays,
their morphology, motility, and phylogenetic distribution of 16S rDNA sequences

| Strain | | | Gram | | Phylogenetic | |
|---|---|---|---|---|---|---|
| No. | Source | Morphology | Stain | Motility | Affiliation | Genus |
| 1 | PVC panel, | Rod | − | + | α- | Rhodovulum |
| | Ponggol | | | | Proteobacteria | |
| | Marina | | | | | |
| 2 | Polycarbonate | Rod | − | 0 | α- | Erythrobacter |
| | panel, | | | | Proteobacteria | |
| | Ponggol | | | | | |
| | Marina | | | | | |
| 3 | Acrylic panel, | Rod | − | 0 | α- | Erythrobacter |
| | Ponggol | | | | Proteobacteria | |
| | Marina | | | | | |
| 4 | Panel, | Rod | + | + | Gram(+) low | Bacillus |
| | Ponggol | | | | G + C | |
| | Marina | | | | | |
| 6 | Cable tie, | Short Rod | − | + | γ- | Vibrio |
| | Ponggol | | | | Proteobacteria | |
| | Marina | | | | | |
| 7 | Oil slick on | Rod | − | + | γ- | Vibrio |
| | rocky shore, | | | | Proteobacteria | |
| | St John's | | | | | |
| | Island | | | | | |
| 8 | Under | Rod | + | + | Gram(+) low | Bacillus |
| | ascidian, | | | | G + C | |
| | Polyclinid on | | | | | |
| | Siglap Buoy | | | | | |
| 9 | Inside tube of | Rod | + | 0 | Gram(+) low | Bacillus |
| | Vermitid, | | | | G + C | |
| | Siglap Buoy | | | | | |
| 10 | Inside sponge, | Rod | + | 0 | Gram(+) low | Halobacillus |
| | Siglap Buoy | | | | G + C | |
| 11 | Under mixed | Rod | − | 0 | γ- | Pseudomonas |
| | fouling | | | | Proteobacteria | |
| | community, | | | | | |
| | Siglap Buoy | | | | | |
| 12 | On yellow | Rod | + | 0 | Actinobacteria | Gordonia |
| | encrusting | | | | | |
| | organism, | | | | | |
| | Siglap Buoy | | | | | |
| 13 | Under sponge, | Rod | + | 0 | Gram(+) low | Bacillus |
| | Siglap Buoy | | | | G + C | |
| 14 | Under | Rod | − | + | γ- | Vibrio |
| | filamentous | | | | Proteobacteria | |
| | algae, | | | | | |
| | Main | | | | | |
| | Fairways | | | | | |
| | Buoy | | | | | |
| 16 | Under | Rod | | + | γ- | Pseudoalteromonas |
| | barnacle, | | | | Proteobacteria | |
| | Main | | | | | |
| | Fairways | | | | | |
| | Buoy | | | | | |
| 17 | Under sponge, | Rod | − | 0 | Actinobacteria | Gordonia |
| | Main | | | | | |
| | Fairways | | | | | |
| | Buoy | | | | | |
| 18 | Under | Rod | − | 0 | Actinobacteria | Microbacterium |
| | bryozoan, | | | | | |
| | Main | | | | | |
| | Fairways | | | | | |
| | Buoy | | | | | |
| 19 | Under red | Rod | − | 0 | α- | Erythrobacter |
| | encrusting | | | | Proteobacteria | |
| | sponge, Main | | | | | |
| | Fairways | | | | | |
| | Buoy | | | | | |

The results of the inhibition and growth data were given in Tables 4-6. The drugs exhibiting strongest activity against the panel of bacteria are ZLT, PZC, TFZ and PCZ. To review the results taking into account the bacteria identification, only PZC and ZLT at the higher concentration of 50 ug exhibited activity against the three Vibrio strains tested. This activity was comparable to activity obtained for the antibiotics. The drugs showed much stronger activity against Erythrobacter strains than commercial antibiotics. These four compounds are bactericidal.

### Example 7

Effect of Pharmaceuticals on Biofilm Formation

Hereinafter, the present inventors report the more detailed results of the studies investigating the effect of pharmaceuticals on bacterial cell adhesion in biofilm formation, and effect of drugs on the integrity of attached bacteria on substrata.

Materials and Method

Biofilm Bacteria

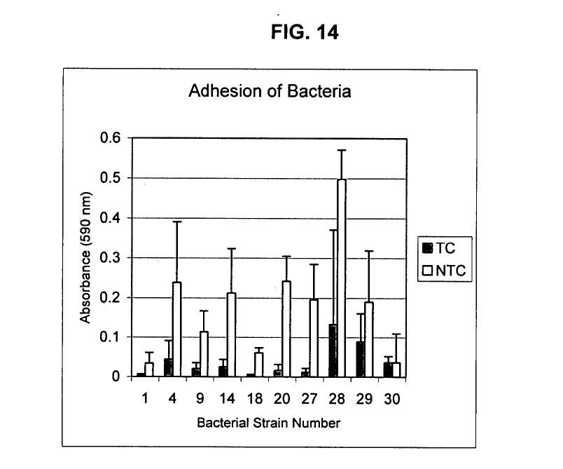

We selected 6 bacteria for analysis, which have been observed to form biofilms on plastic in the laboratory (FIG. 14). This includes a motile and non-motile Bacillus, and four other common genera we have found associated with tropical marine biofouling. These bacteria will form a biofilm after two hours on laboratory polystyrene. In general, the bacteria prefer untreated polystyrene to tissue culture treated polystyrene which is hydrophilic.

Test Pharmaceuticals

The eight pharmaceuticals tested were: CYZ, ZFN, ZPX, IMD, HDL, PZC, ZLT, TFZ, PCZ. The drugs PZC, ZLT, TFZ, PCZ have strong bactericidal activity.

Pharmaceutical Preparation

Commercial forms of pharmaceuticals were used as previously described. A tablet with a known amount of active ingredient (as stated in the product information) was ground in a mortar and pestle and suspended in sterile deionized water (SDI) to a concentration of 2 mg ml⁻¹. These samples were sonicated for at least 1 min and these fine suspensions were used as initial stock solutions.

For studies of bacterial adhesion, the pharmaceuticals were first sterilized by soaking in absolute alcohol (Merck) for half an hour. Ethanol was removed by air-drying with air passed through 0.25 μm pore size syringe filter (Sartorius, Germany). Stock solutions were concentrations of 100 μg ml−1 in SDI.

Effect of Pharmaceuticals on Substratum Wettability

It was recognized at the onset that the drugs may modify the surface properties of the plastic, thereby changing the behavior of the bacteria. To determine the extent of surface modification resulting from the drugs, we measured change in surface wettability using the Standard Harmonic Mean (SHM) method developed by Gerhart et al. (1992).

As it was not possible to measure the SHM in the small wells of the 96-well plates, we searched for petri dishes made of the same material. We found 35 mm diameter cell culture dishes (Falcon 353001) are manufactured from similar materials as the tissue-culture treated 96 well plates (Falcon 353072) (personal communication, Biomed Diagnostics Pte Ltd., Singapore). The base of 35×10 mm diameter sterile Petri dish (Falcon 353001) is tissue-culture treated polystyrene while the cover is untreated polystyrene.

In each polystyrene dish cover, 3 ml of each pharmaceutical in seawater at concentrations of 25 and 50 μg ml⁻¹ were incubated for 2 h, while in the dish bottom only 1 ml of pharmaceutical solutions was used. After 2 h, the dish covers and bottoms were rinsed with SDI water and air dried. Controls consisted of untreated dish covers and bottoms. Once dried, 25 μl drops of a series of solutions of SDI and methanol were placed onto the surface to measure the diameter of the drop-spread to the nearest mm. The final value was the average of three droplets for each solution. The solutions included 100, 80, 60, 40, 30, 20, 10, and 0% water in HPLC grade methanol (Lab Scan Analytical Lab). The whole experiment was repeated three separate times. The values were used to calculate the SHM of the drop measurements according to the formula presented by Gerhart et al. (1992).

Effect of Pharmaceuticals on Adhesion of Bacteria to Plastic

The effect of pharmaceuticals on bacterial adhesion was analyzed using a modified microplate method adapted from Shea and Williamson (1990). This method employs a combination of staining attached bacteria with crystal violet, releasing the stain with sodium desoxycholate, and measuring the resulting absorbance of the crystal violet. Shea and Williamson (1990) demonstrated a positive correlation between the number of colony-forming units (CFU) and the resulting absorbance.

The adhesion of ten strains of bacteria used in adhesion experiments has been presented in Table 8 above. Detailed results for six selected strains are presented here: strains 4, 9, 14, 20, 27, 28. The bacterial strains were grown in Marine Broth (Pronadisa, Spain) overnight, in the dark, at 28° C. on a rotary shaker (150 rpm) in 5 ml centrifuge tubes (Falcon). Cells were harvested by centrifugation (1500×g, 10 min) and resuspended in sterile seawater (SSW). SSW was prepared by filtering seawater through a 0.2 μm pore size filter (Sartorius) followed by autoclaving. The cells were centrifuged and resuspended once more in SSW. The concentration of resuspended cells was adjusted to A₆₀₀=1.0 using a spectrophotometer (Beckman DU530, USA).

Flat bottom 96 well polystyrene (hydrophobic, Falcon 351172) (=NTC) and tissue-culture treated polystyrene (hydrophilic, Falcon 353072) (=TC) microplates were used in the analyses. Each test was performed in triplicate. Two sets of experiments were conducted. The first set had a final concentration of 25 μg ml⁻¹ of the respective pharmaceutical. For the control and blank wells, 25 ul of SDI were added. For wells containing pharmaceuticals, 25 ul of the respective pharmaceutical at a concentration of 100 ug ml−1 were added. This was immediately followed by the addition of 75 ul of bacterial suspension. To blanks were added 75 ul of SSW.

The bacterial cells in suspension were allowed to attach for 2 h at 28° C. Unattached cells were removed by aspiration using a plate washer (Tecan, Austria) and wells were rinsed three times with 200 ul of SSW each time. The attached cells were stained with 200 ul of crystal violet solution (0.1% w/v in deionized water) in each well for 5 min. After removing the stain using the plate washer, the wells were rinsed twice with 300 ul of SDI each time. Sodium desoxycholate (200 ul) solution (2% w/v in deionized water) was added to each well to extract the crystal violet from the attached stained cells for 10 min. The absorbance of the crystal violet was read at 590 nm using a microplate reader (Sunrise, Tecan, Austria).

Results of the adhesion assay were obtained by the difference in absorbance readings between wells containing bacterial cells and the mean of absorbance of those containing the SSW only. The effect of the pharmaceuticals on bacterial adhesion was determined by the examining the differences in absorbance between the wells containing the pharmaceuticals+bacteria and the bacteria. The significance was compared using T-test.

Effect of ZPX and ZFN on Adhesion and Removal of Biofilms

Additional experiments were carried out for ZPX and ZFN to determine the effect of ZPX and ZFN on the bacteria, when they are added before or after biofilm formation on NTC surfaces.

The bacteria were grown, washed and their concentrations were adjusted as described above. The bacterial suspension (75 μl) of strains 9, 14, 20, 27 and 28 in SSW and 50 μl of SDI water were placed into 96 well polystyrene microplates (Falcon 351172). Blank wells were filled with 75 μl of SSW and 50 μl of SDI. Three wells were used for blanks, each control and treatment.

The cells were allowed to attach to the wells for 2 h. Afterwards, unattached bacteria and liquid in the wells were removed by aspiration and wells were washed with 200 μl of SSW twice using a microplate washer as described above. After washing, 75 μl of SSW were added and 50 μl of SDI to the control (bacterial suspension and SDI as above) and blank (75 μl of SSW and 50 μl of SDI) wells. For wells with ZPX and ZFN treatments, 75 μl of SSW and 50 μl of the pharmaceuticals at a concentration of 125 μg ml−1 (final concentration 50 μg ml−1) were added.

After 2 h, the wells were washed with SSW, washed twice with SDI and stained with crystal violet as described above. After 10 min, the stain was removed, wells were washed twice with SDI, and crystal violet was released from the attached bacteria by adding 200 μl of sodium desoxycholate. After 10 min, absorbance (A₅₉₀) of each well was read using a plate reader (Tecan Sunrise, Switzerland). Comparisons were made between wells where attached bacteria were exposed to SDI and wells where they were exposed to the pharmaceuticals.

Results

Adhesion to Untreated Substrata

The adhesion of the different strains of bacteria to TC and NTC are given in FIG. 14. The 6 selected bacteria (strains 4, 9, 14, 20, 27, 28) showed differences in their adhesion to TC and NTC surfaces, with greater preference for NTC to the hydrophilic TC plates. Hence, in the main experiments with the pharmaceuticals, only data for strains 4, 14 and 28 in experiments on the TC plates is presented, as they produced a significant biofilm on these surfaces.

Effect of Pharmaceuticals on Surface Wettability

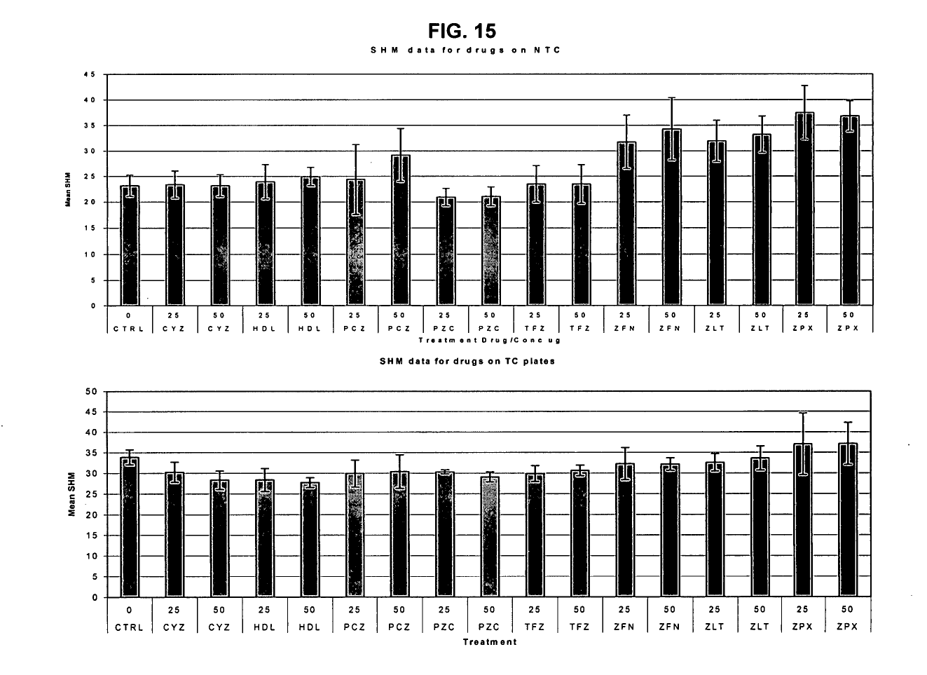

The results are given in FIG. 15. Tissue-culture treated Petri dishes gave a SHM reading of 33.9 while the untreated polystyrene had a SHM of 23.2. The tissue culture plates are hydrophilic surfaces and have a higher wettability coefficient. Most of the drugs reduced the wettability of the tissue culture plates, with the exception of ZFN and ZLT. ZPX, ZFN, ZLT significantly increased the wettability of untreated polystyrene, whilst PZC and PCZ gave a slight increase in wettability. HDL, CYZ and TFZ had little effect on the untreated polystyrene.

For purpose of this study, we assumed that any significant change in SHM translates to surface modification in way of adsorption of drugs to the surface of the plates. From the SHM measurements, we conclude that most of the drugs adsorb to the different plastics to different degrees.

This effect may be a confounding factor in the adhesion assay. We assumed that bacteria preference was a result of surface wettability as biofilm formation is known to be affected by surface wettability (Absolom et al., 1983; Dexter et al., 1975). If that is the case, as our bacteria prefer more hydrophobic surfaces to the hydrophilic tissue-culture plates, we expect that the drugs would increase bacteria preference for the tissue culture plates as they decreased the wettability of these plates. In the same way, increasing the wettability of the untreated polystyrene may result in a reduced preference.

However, the adsorbed compounds can act in way of a direct chemical effect on the bacteria, which may be attractive or repellent. Hence, reduced biofilm formation may be a result of (1) bacteria no longer prefers the surface as a result of change in wettability arising from the adsorbed chemicals; (2) adsorbed molecules are toxic and repellent to the bacteria; and (3) drugs in solution act on bacteria cells, either killing or affecting them in such way that they cannot form a film on the surface. The latter two effects are relevant for antifouling purposes.

Effect of Pharmaceuticals on Adhesion

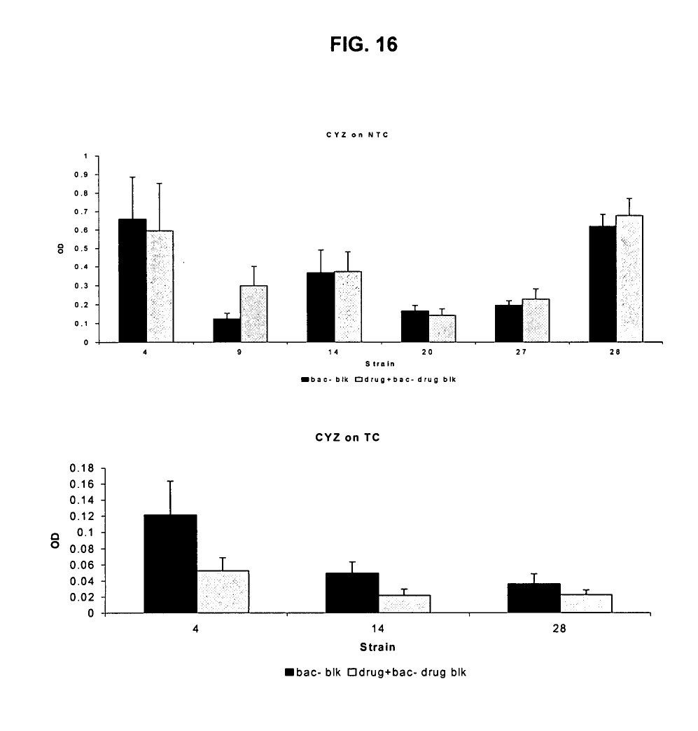

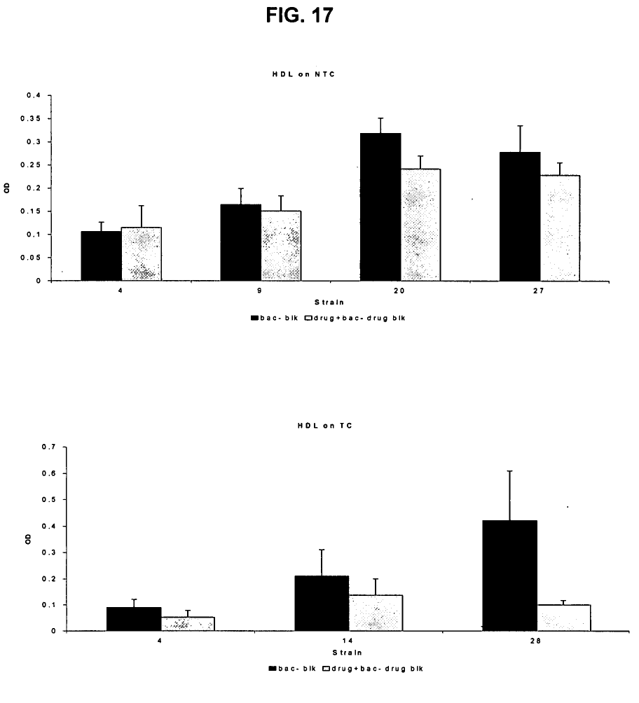

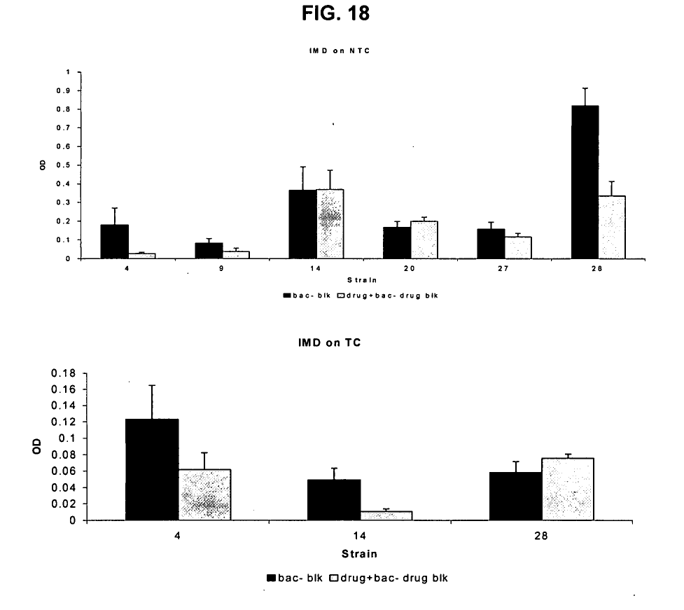

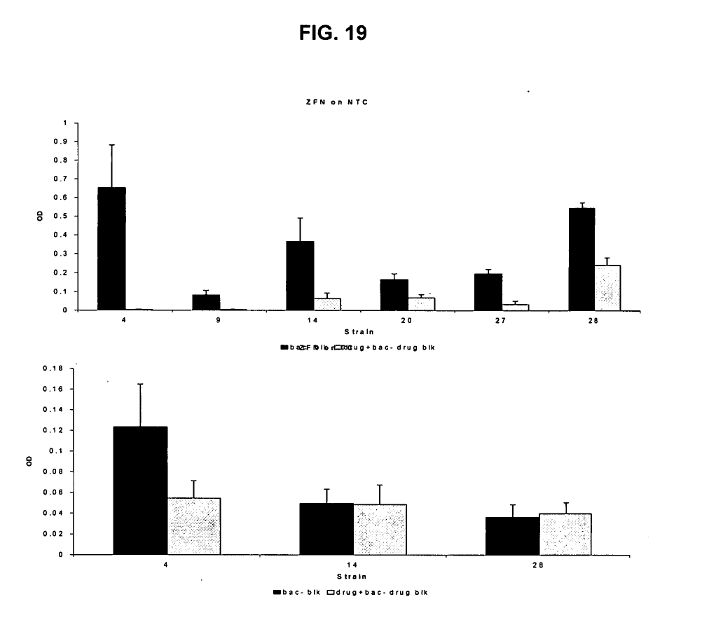

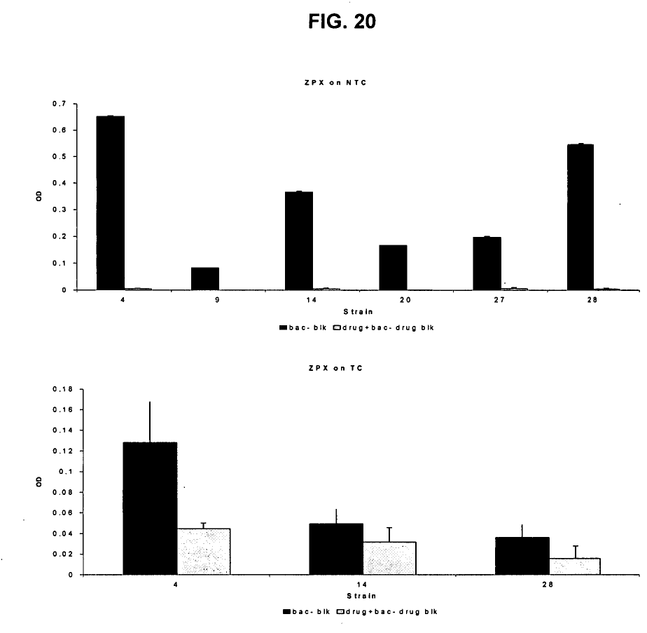

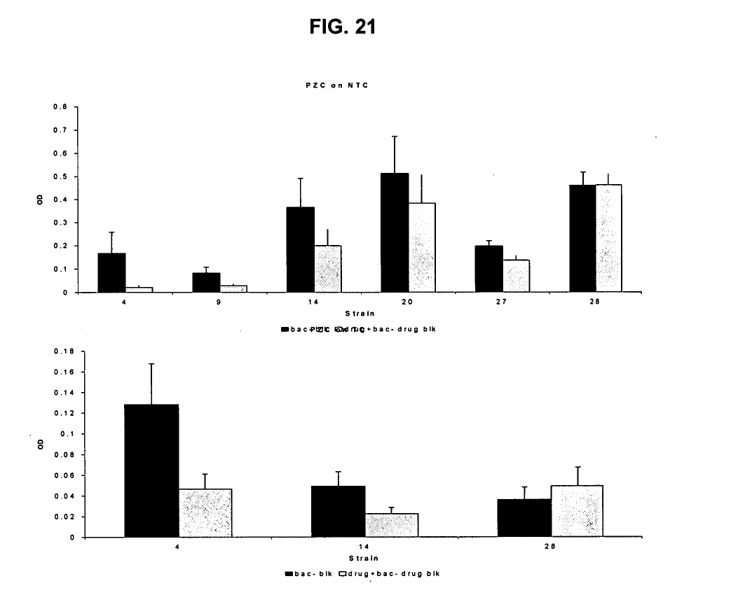

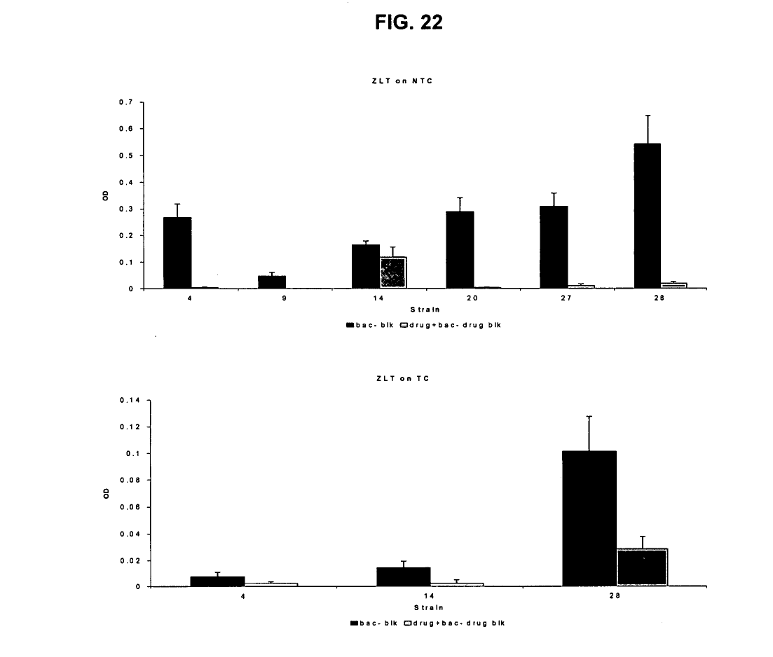

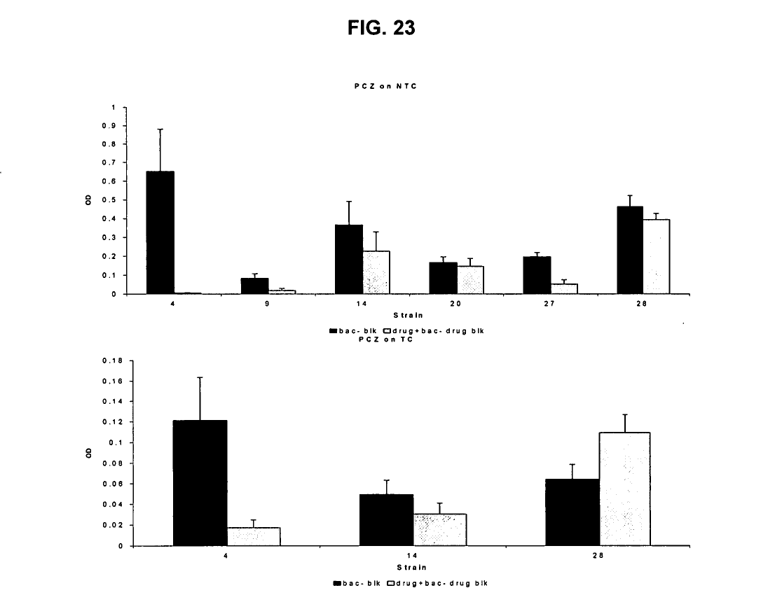

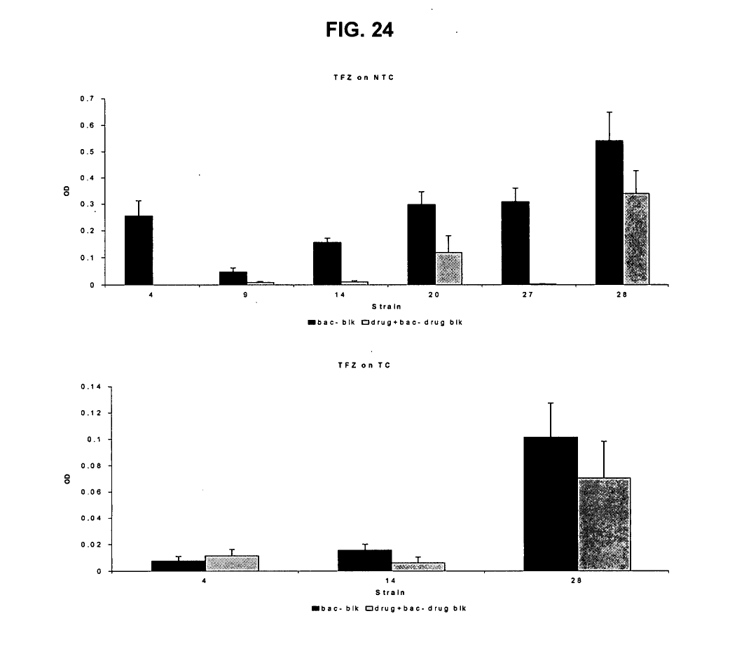

The results for the different compounds tested at 25 ug ml⁻¹ is given in the FIG. 16-24. The pharmaceuticals were also tested at 50 ug ml⁻¹ concentration and the general trends obtained were similar.

CYZ: There was significantly more bacteria adhesion for strain 9.

HDL: There was no significant difference from control for these 4 bacteria. On TC, there was a slight reduction in biofilm formation for strain 28 (p<0.1).

IMD: On NTC, there was a slight reduction in strain 28.

ZFN: ZFN reduced bacterial adhesion of the 6 strains to the NTC plates. We know from the SHM experiments that ZFN will adsorb to NTC. However, adsorption to TC was not significant. There was only slight activity (p<0.1) against strain 4 on the TC plates. ZFN appears to have surface-active properties.

ZPX: From the SHM tests, we know that ZPX adsorbs to both NTC and TC plates. ZPX prevented biofilm formation for all 6 strains in the NTC plates, and slightly reduced the biofilm formation in strains 4 on the TC plates.

PZC: We have observed bactericidal activity against the 6 strains, for this drug. From the SHM data, we conclude that some amount of PZC will adsorb to TC and NTC but the change in SHM is not very marked. PZC appears to have slight effect on biofilm formation in strain 4 on both TC and NTC plates.

ZLT: ZLT is a powerful bactericide. Biofilm formation was reduced for most strains except strain 14.

PCZ, TFZ: These 2 compounds are structural analogues. Both adsorb to TC but only slight change was observed in the SHM data for the NTC plates. Both compounds exhibited bactericidal activity against the 6 strains in earlier experiments. TFZ is a more powerful antibiotic than PCZ. PCZ had reduced biofilm formation only for strains 4 and 27. TFZ reduced biofilm formation on NTC plates.

Effect of ZPX and ZFN on Adhesion and Removal of Biofilms

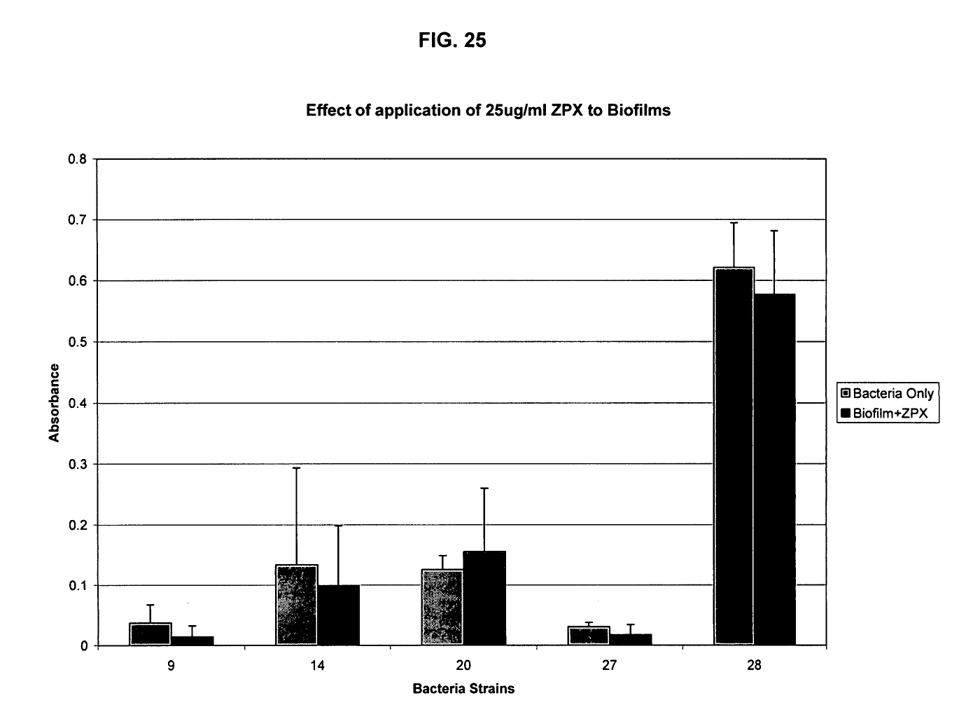

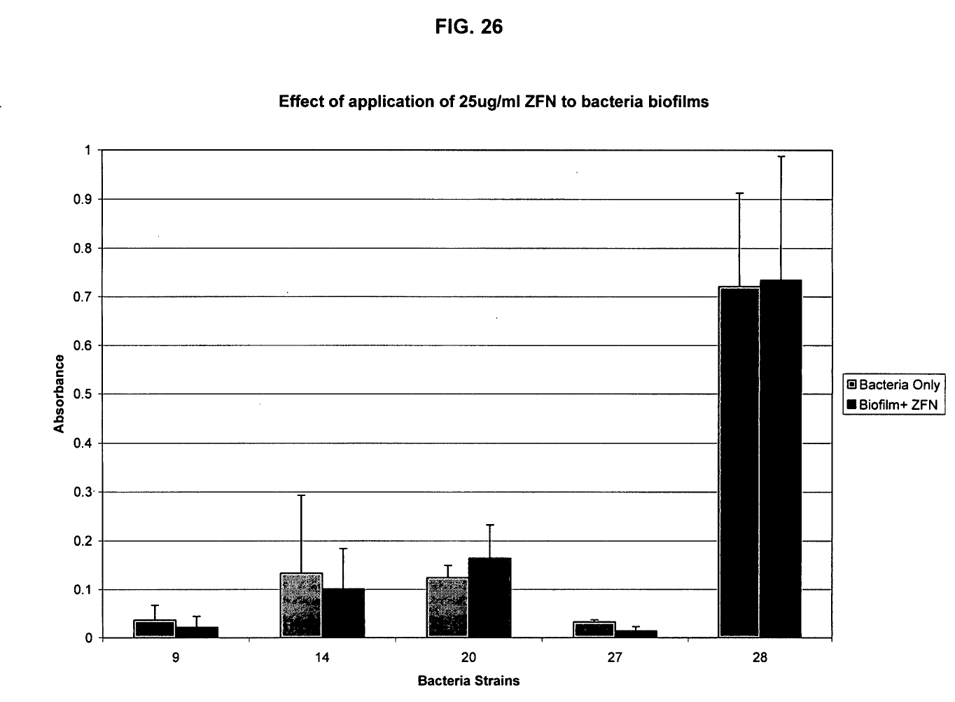

When ZPX, ZFN were applied to established bacterial biofilms at 25 ug ml⁻¹, there was no significant effect (see FIGS. 25, 26) even though, as described above, the bacteria were unable to form biofilms in the presence of ZPX and ZFN.

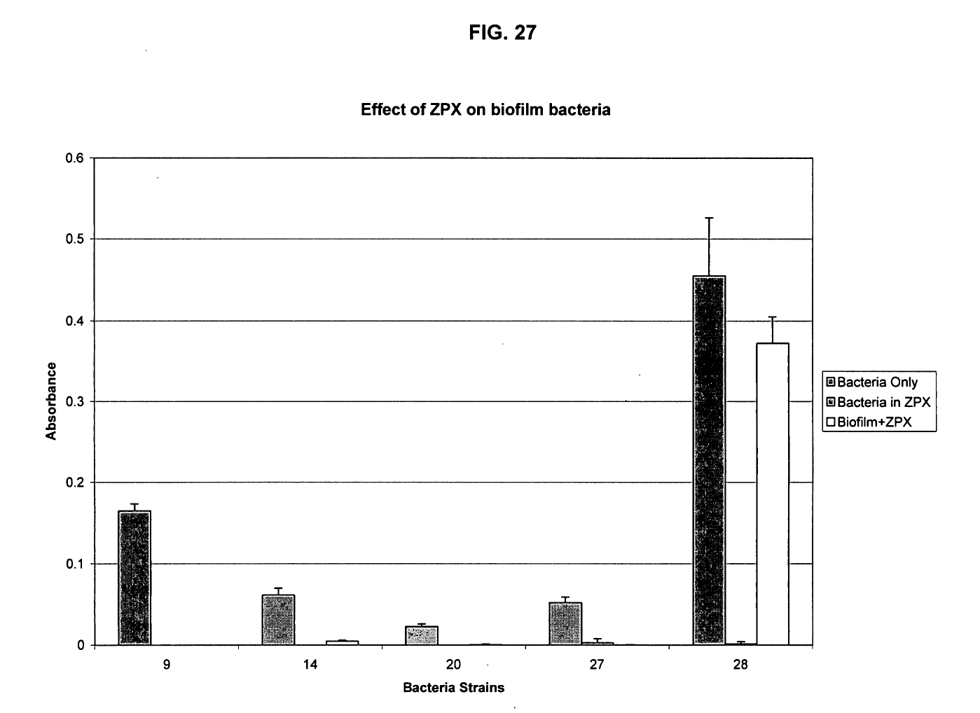

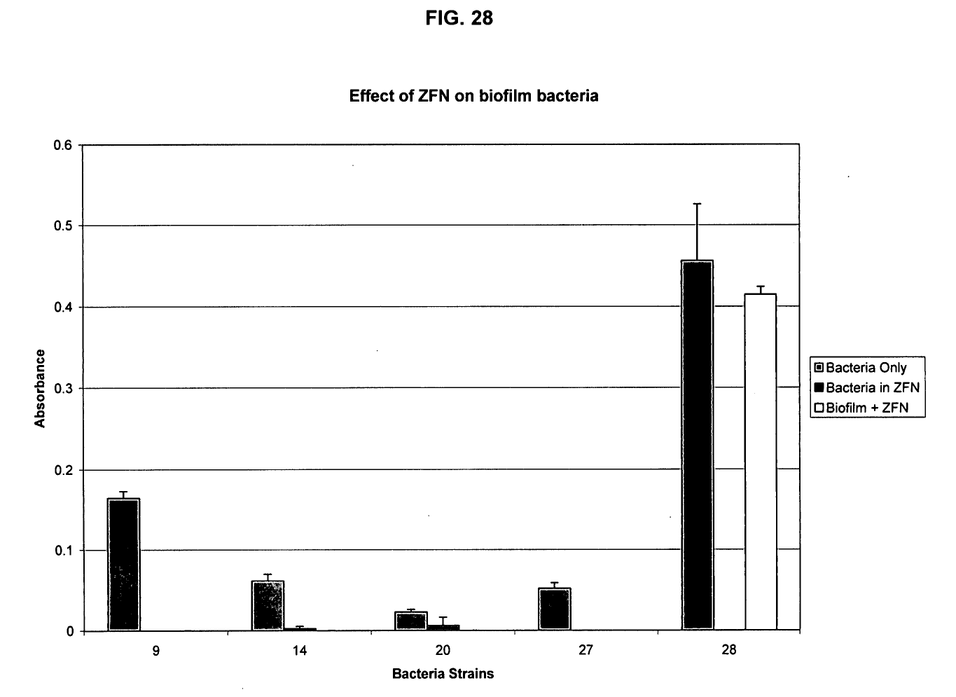

However, at 50 ug ml⁻¹ concentration of drug (FIGS. 27, 28), interesting activity was observed. Except for strain 28, little or no biofilm was present after treatment with ZPX, ZFN whether or not the drugs were added together with the bacteria, or added after a biofilm was established. In the case of strain 28, the drugs are able to prevent the formation of a biofilm.

ZPX and ZFN have strong effects against biofilm formation at 50 ug ml⁻¹. At 25 ug ml⁻¹, the drugs can prevent formation of a biofilm.

Discussion (for Examples 6 and 7)

Although surface wettability may be a confounding issue, the marked decrease in adhesion resulting from treatment with some of the compounds indicate that adsorbed molecules of these drugs are useful in preventing bacterial settlement and biofilm formation. ZPX, ZFN and the bactericide ZLT have potential application for reduction of biofilm formation.

The presence of adsorbed molecules of these drugs alter biofilm formation on surfaces. This has important implications for antifouling, as presence of these compounds in a material can alter the structure of the biofilms on the materials. On one hand, these compounds can have applications in reducing microfouling on surfaces. On the other hand, from the perspective of a biofouling community, species-specific effects on bacterial biofilm formation are as important as specific bacteria consortia have effects on subsequent macrofouling.

Summary of Results from Bacteria Studies

ZPX, ZFN and ZLT have unusual anti-bacterial properties against marine biofilm bacteria. ZLT demonstrated strong bactericidal activity. ZFN and ZPX have unusual activity against biofilm formation. These 3 compounds have application as antifoulants.

Effect on Larvae of Macrofoulers

Effects of the compounds against larvae of microfoulers were demonstrated as follows.

Preparation of Pharmaceuticals from Commercial Tablet

Pharmaceuticals from the respective manufacturers were treated in the following way. A pill containing a known amount of active ingredient (as stated in product information) was ground in a mortar and pestle and a suspension of 2 mg ml⁻¹ generated in de-ionized water. This stock solution is stored at −20° C. in 4 ml amber screw cap vials. For the bioassays, 250 ul of stock solution is added to 10 ml of 1 um filtered seawater. This suspension was then sonicated for 10 minutes. Serial dilutions of this 50 μg ml⁻¹ seawater solution, or suspension, with seawater was done to generate the required range of concentrations. As only small amounts of stock solution were added to sea water which contains milli-molar amounts of carbonate and bicarbonate that buffer pH and milli-molar amounts of salts, there was no significant change in pH or salinity of the test solutions as compared to the control.

### Example 8

Barnacle Toxicity Assays

Toxicity assays were modified from Rittschof et al. (1992.a). Stage II naupliar larvae used in tests were obtained from Balanus amphitrite adults collected from inter-tidal rock walls at Kranji mangrove, Singapore. Larvae were collected from a container of adults by attraction to a point source of light and transferred to 500 ml of fresh seawater. Next, larvae were re-concentrated with a fiber optic light and added to assays.

Duplicate assays were conducted in 2 ml micro-centrifuge tubes (Axygen MCT-200-C) in 1.5 ml of filtered seawater for 22 to 24 hours. The assays were repeated for confirmation. There were four controls (vehicle only, 0 μg ml⁻¹), and 2 tubes of each for each test concentration. The assay was initiated by addition of barnacle nauplii in 50 to 100 μl of seawater.

After 22 to 24 hours of incubation at 25-27° C., solutions containing test animals were transferred to a Bogorov tray and scored as living or dead. Moribund larvae were scored as dead. Results were confirmed by repeating the assay. Data were combined and the concentration that caused 50% mortality (LC₅₀) was calculated by probit analysis using a basic computer program (Libermann, 1983). If data were not appropriate for probit analysis, the LC₅₀ was estimated from graphed data. The results are given in Table 11.

### Example 9

Barnacle Settlement Bioassay

The bioassay method used follows from the method first introduced by Rittschof et al. (1992), and subsequently by other authors (Willemsen et al., 1998). This method is now standard practice for many antifouling screening of novel compounds.

Barnacle larvae from field-collected adults were reared on an algal mixture of 1:2:2 v/v of Rhodomonas salina, Tetraselmis suecica and Chaetoceros muelleri at 25° C., at approximately 5×10⁵ cells per ml density. On this regime, larvae metamorphose to cyprids in 5 days. These cyprids were aged at 4° C. for 4-5 days, and 45-70% settled on perspex after 24 hours (Willemsen et al., 1998).

Settlement tests were conducted in 24 well culture plates (Cellstar # 662160), with 20-40 cyprids per well. Test solutions were made up to twice the required final concentration. 1 ml of test solution was added into each well, and cyprids were transferred into each well in 1 ml of seawater. Assays were done in triplicate. After 24 hours, larvae that had attached and metamorphosed were enumerated and the result expressed as percentage settlement. Larvae not attached were scored as not settled. The assays were repeated and the concentration that caused 50% settlement inhibition (EC50) was determined by probit analysis using a basic computer program (Lieberman, 1983), or by estimation from graphed data. The results are given in Table 11.

Therapeutic Ratio

The Therapeutic Ratio, or LC₅₀/EC₅₀, is a way of assessing the effectiveness of the compound in relation to its toxicity. Therapeutic ratios less than one are indicative of compounds that are toxic. Therapeutic ratios>>1 are indicative of compounds where the concentration that will prevent settlement is non-lethal. These compounds are potentially useful as environmentally benign antifoulants. LC₅₀ and EC₅₀ estimates and the therapeutic ratio should be considered as working values providing an estimation of potency, rather than as absolutes.

TABLE 11
Results of barnacle bioassay

| Drug | LD50 | | ED50 | | Therapeutic | |
|---|---|---|---|---|---|---|
| reference | (ng ml-1) | | (ng ml-1) | | ratio | Notes |
| CYZ | 61.05 | | 155.70 | | 0.39 | |
| HDL | 252.68 | | 119.16 | | 2.12 | |
| IMD | 241.34 | | >5000 | | <0.05 | |
| PZC | 139.41 | | 1973.84 | | 0.07 | |
| PCZ | 3.54 | | 173.65 | | 0.02 | |
| TFZ | 9.35 | | 74.82 | | 0.12 | analogue |
| | | | | | | of PCZ |
| ZFN | 468.96 | | 241.69 | | 1.94 | |
| ZLT | 1.58 | | 501.85 | | 0.003 | |
| ZPX | 505.82 | | 2.31 | | 218.97 | |
| TBT | 3.4 | ng/ml | 0.09 | ppm | 0.05 | Willemsen |
| | | | 66 | ng/ml | | et al., 1998 |
| | | | | | | Rittschof, |
| | | | | | | 2000 |

| | | | | | |
|---|---|---|---|---|---|
| Sea | — | 0.33 | ppm | — | Willemsen |
| Nine | | | | | et al., 1998 |

Summary of Results (Examples 9-10)

Most of the compounds exhibited strong anti-larval effects, with LD₅₀ and/or ED₅₀ values well below 1 ug ml⁻¹. All the compounds were toxic to larvae at concentrations above 1 ug ml⁻¹.

CYZ and HDL were toxic to barnacle nauplii and cyprids. PCZ and its analogue TFZ were both very toxic to nauplii and cyprids. These compounds have potential application as biocides.

For IMD, the LD₅₀ against nauplii was low but the compound was not effective against cyprids. There may be confounding factors due to poor solubility of the drug and drug delivery issues involved.

PZC had a relatively low LD₅₀ but the ED₅₀ was not impressive. ZLT had a very low LD₅₀ but moderate low ED₅₀.

ZFN gave moderately low LD₅₀ and ED₅₀, with a therapeutic ratio greater than 1, suggesting potential application as a repellent.

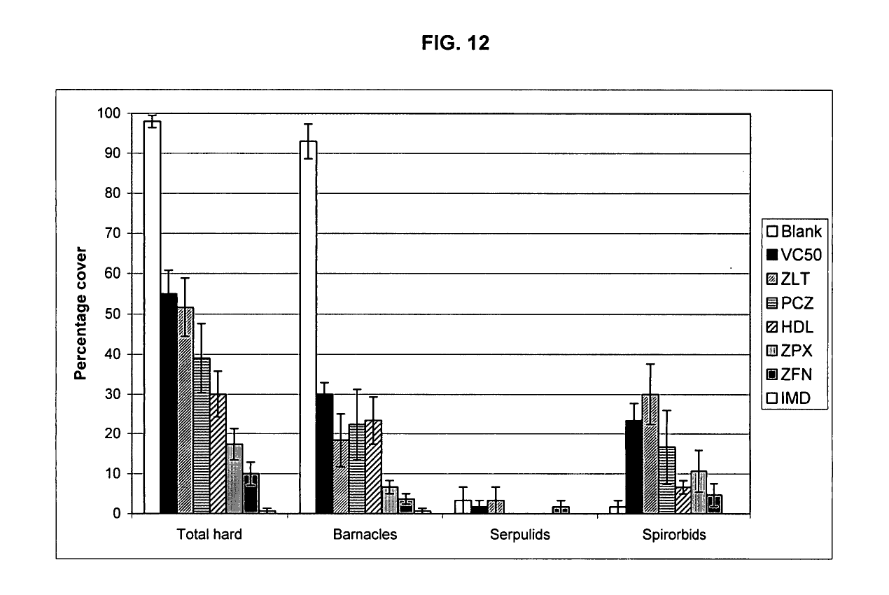

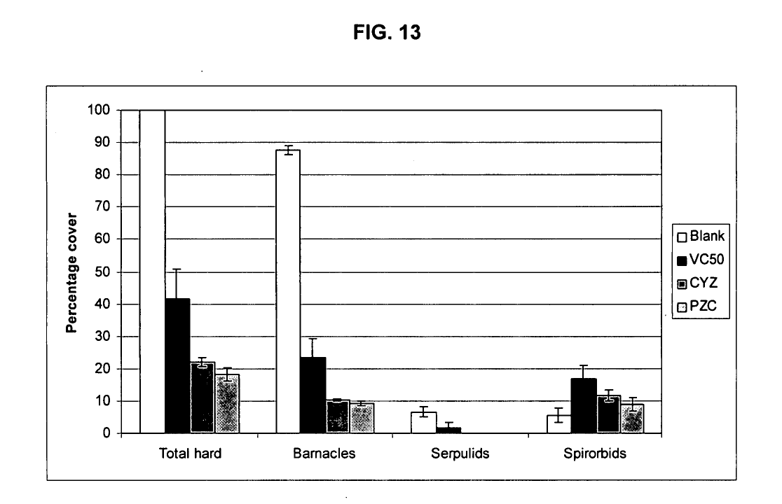

Of particular interest is ZPX which has a very high therapeutic ratio. The compound had a very low ED₅₀ value. FIG. 12 gives more detail on the some of the settlement assay results. It is noted that at concentrations less than 1 ug ml⁻¹, the cyprids were not killed but were not able to settle.

### Example 10

Bioassay Against Bryozoan Larvae

Bugula neritina (Phylum Bryozoa, Class Gymnolaemata, Order Cheilostomatida) is a cosmopolitan species of bryozoan, commonly found in most marine fouling communities. The larvae are brooded in ovicells found on the stem of the colonies. When released, the larva will settle quickly on most surfaces, within a few minutes. At 24° C., the settled larvae forms the one-cell ancestrula, and after 48 hours, the lophophore has developed and extended for feeding.

Collection of Adults and Spawning for Larvae

Adult colonies of Bugula neritina were collected from fouling communities growing on the submerged pontoons on a floating fish farm located off Changi, Singapore. Adult animals were maintained in the open-circulation aquarium at St John's island marine laboratory, Singapore. The adults were kept in total darkness for 24 hours. On exposure to light, larvae were released after half an hour. Larvae were collected from a container of adults by attraction to a point source of light. Because the larvae will settle quickly, larvae were pipetted directly into test wells in 1 ml of seawater using a graduated glass pipette.

Experiment

Settlement tests were conducted in 24 well culture plates (Cellstar # 662160), with 10-20 larvae per well. Test solutions were made up to twice the required final concentration. 1 ml of test solution was added into each well, and larvae were transferred into each well in 1 ml of seawater. 4 replicates were done for each treatment. After 24 hours, larvae were scored as settled, swimming or dead/deformed. The assay plates were usually kept for another 24 hours and checked again, to ascertain that the settled larvae had developed into viable colonies with lophophores. Two trials were repeated for each compound.

Bioassays were carried out using the method described above, in July 2003 during the peak spawning season for Bugula.

In addition, an additional drug, Aurorix® (containing the active ingredient moclobemide) was tested against Bugula larvae. No activity against Bugula neritina (or other larvae, Balanus amphitrite) was detected for all concentrations tested, even at 25 ug/ml. The tablet produced a murky suspension in seawater. This did not produce any appreciable effect against the Bugula larvae, and healthy, normal lophorate individuals were obtained, even after 96 hours in the test suspension, indicating that the Bugula larvae were not appreciably affected by the reduced water quality arising from the test suspensions.

Results

TABLE 12
Activity on drugs against Bugula larvae

| Drug Ref | Estimated ED50 (ng/ml) |
|---|---|
| CYZ | 2132 |
| HDL | 1128 |
| IMD | 3126 |
| PCZ | 567 |
| PZC | 200 < ED < 1000 |
| ZFN | 1000 < ED < 10,000 |
| ZLT | ED < 200 |
| ZPX | 1000 < ED < 5000 |

The results are summarised in Table 12. As the larvae settled very quickly, they were not very responsive toward small changes in drug dose. Hence, it was not always possible to resolve the data with probit and obtain an ED₅₀ value. Overall, Bugula larvae are less sensitive to the drug compounds than barnacle larvae.

ZLT was the most toxic to larvae, with an ED₅₀<200 ng ml⁻¹. This is followed by PZC, PCZ and HDL with ED₅₀s in the range of 200-1000 ng ml⁻¹; then ZPX, CYZ IMD and ZFN, with ED₅₀s in the range 1000-5000 ng ml⁻¹.

Discussion (for Examples 8-10)

For novel natural products, activity below 25 ug ml⁻¹ is regarded as having potential commercial value. Table 11 also provides some reported LD₅₀/ED₅₀ of some commercially available biocides. Given that the drugs tested are not in pure form and hence drug delivery into the test solutions is not optimised, the values we have obtained for the above assays confirm that the drugs are extremely potent and suggest strong commercial potential for development as antifouling biocides.

### Example 11

Activity of IMD in Plastic and Glass

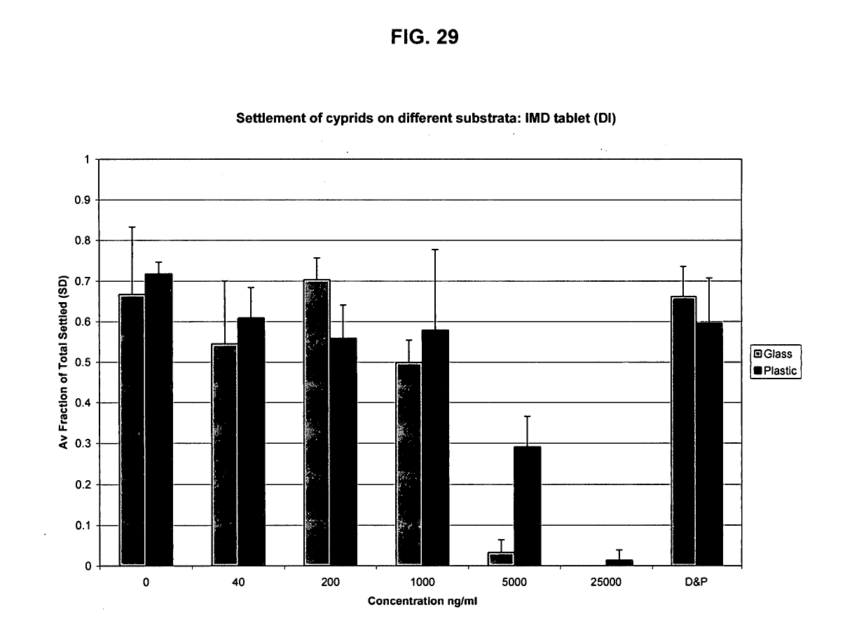

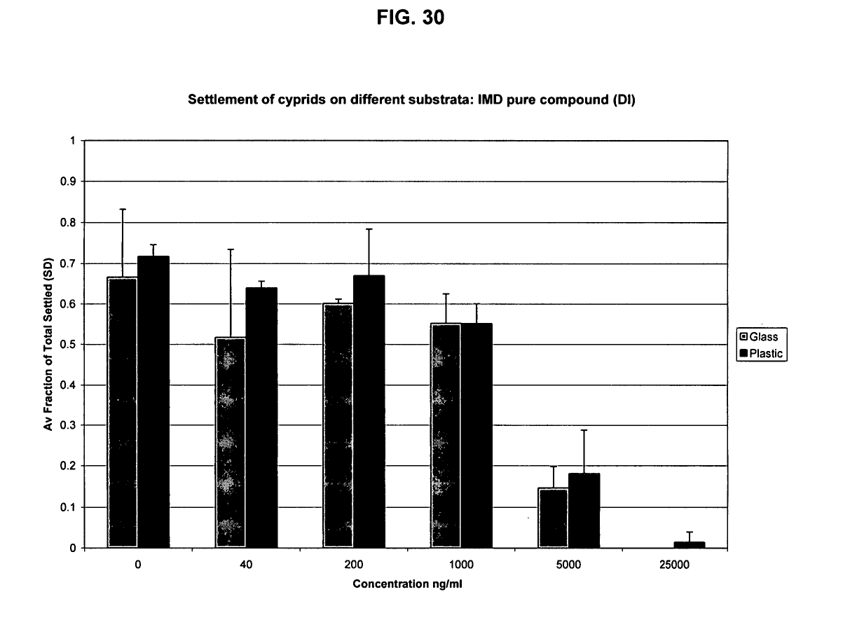

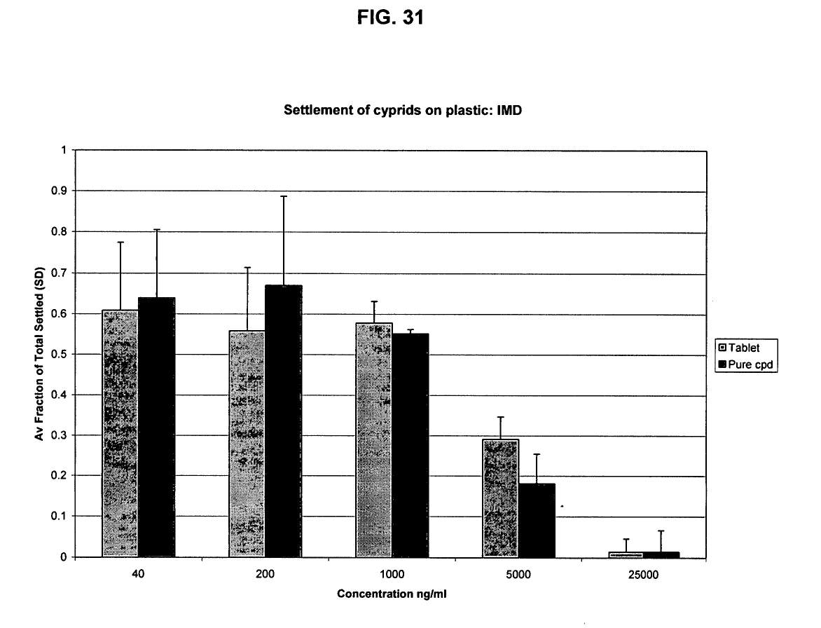

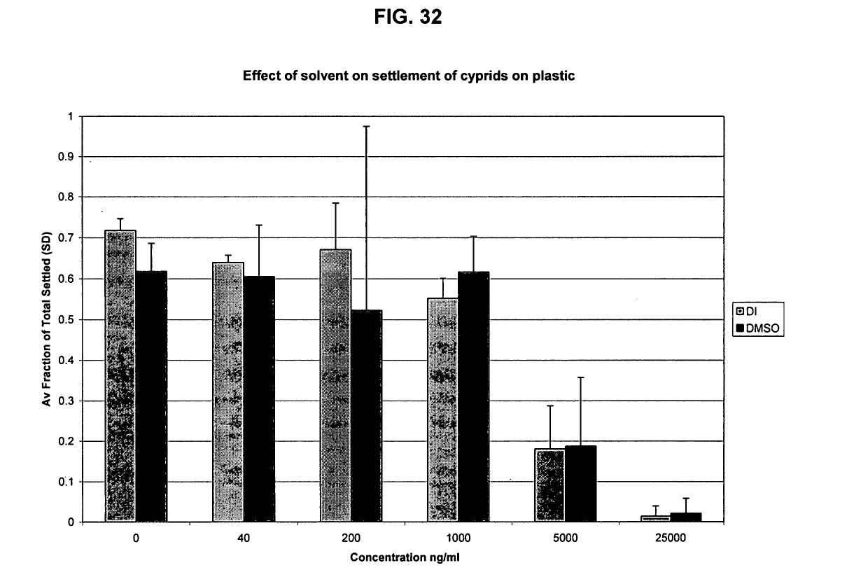

An assay was carried out to attempt to examine the solubility issues for IMD, consisting the following treatments:
(i) IMD pure compound stock solution was prepared using DMSO instead of deionised water. Assays were then carried out on plastic and glass substrata. IMD pure compound (Loperamide hydrochloride) was purchased from TCI Tokyo Kasei Company (Product Ref L0154).
(ii) IMD tablet stock was tested in plastic and glass substrata. In addition, some wells were treated with IMD at the highest concentration 25 ug/ml for 10 mins. The wells were then rinsed with clean water, and cyprids added in clean seawater.
   The results are given in FIGS. 29-32. There was no difference in activity between the solutions prepared from tablet and the pure compound; and no difference between the DMSO and DI prepared stock solutions. There was also no difference between glass and plastic. The results for the treated wells (D&P) also indicate that there was no significant IMD residue present on the surfaces.

### Example 12

Activity of ZPX in Plastic and Glass

From the SHM data above for polystyrene and tissue culture polystyrene (see section on bacterial adhesion) we hypothesized that the repellent effect observed for ZPX in earlier settlement experiments may be a result of interaction of ZPX with the plastic.

A follow up experiment was carried out to examine the effect of ZPX on barnacle settlement when the experiment is carried out on different substrata. ZPX test solutions were prepared as described above from the tablet. The following test conditions were applied:
(1) Assay was carried out with ZPX test solutions in the 24 well tissue culture plates (as before);
(2) ZPX test solution at 25 ug ml⁻¹ were applied to the 24 well tissue culture plates and left in the well for 10 minutes. The solution is then discarded and the well was rinsed three times, each time with 2 ml of seawater. Clean seawater was added into each well.
(3) The assay with ZPX test solutions was carried out in glass scintillation bottles.

Cyprids were obtained and settlement experiments were carried out as described earlier. After 24 hours, the number of cyprids which had settled/attached was counted.

Results

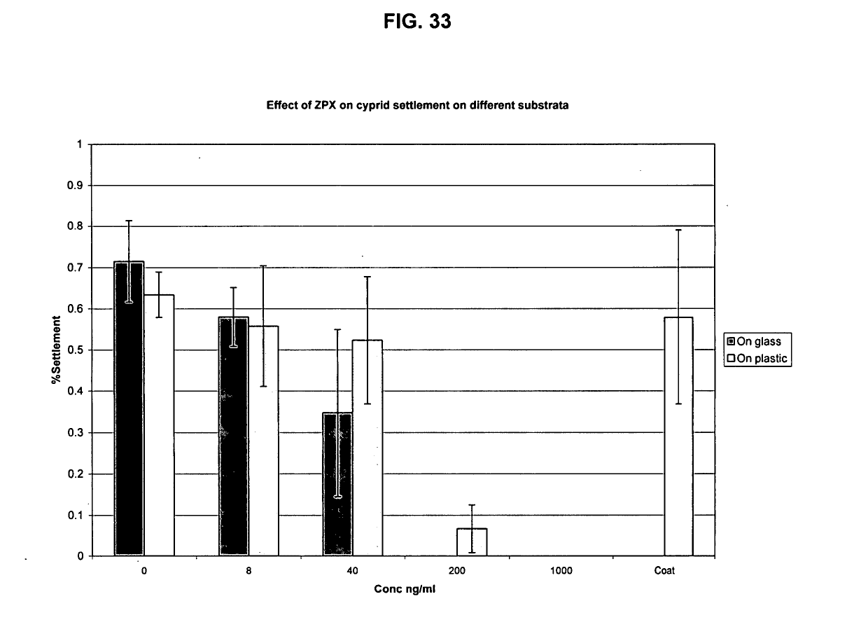

The data is presented in FIG. 33. There was no significant difference in settlement between plastic and glass. After rinsing, the effect of ZPX in plastic was removed and settlement in the “ZPX-coated” well was the same as in the control. Hence, it was concluded that the surface modification effect of ZPX on polystyrene was not a significant factor in the earlier results. ZPX prevents the settlement of barnacle cyprids.

### Example 13

Toxicity of Drugs Under Different Light Conditions and Over Time

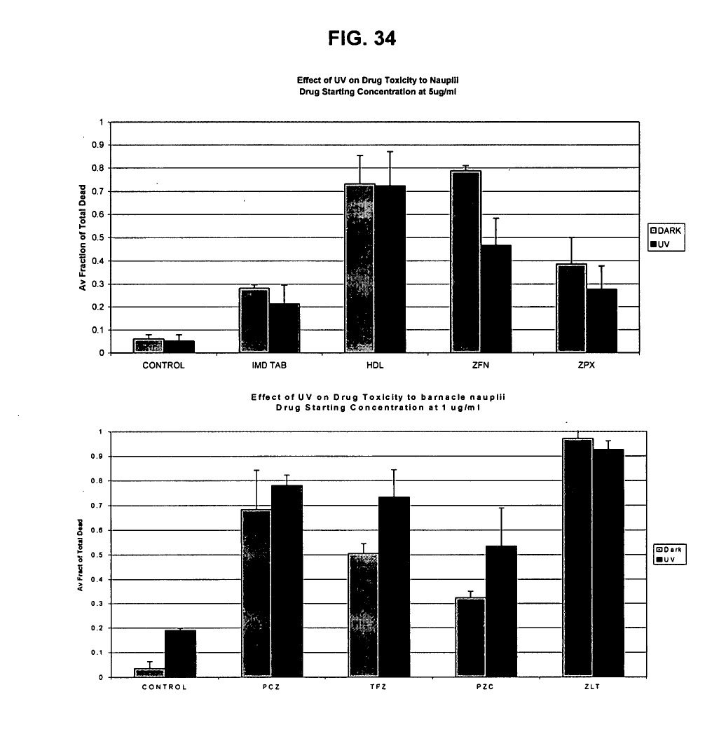

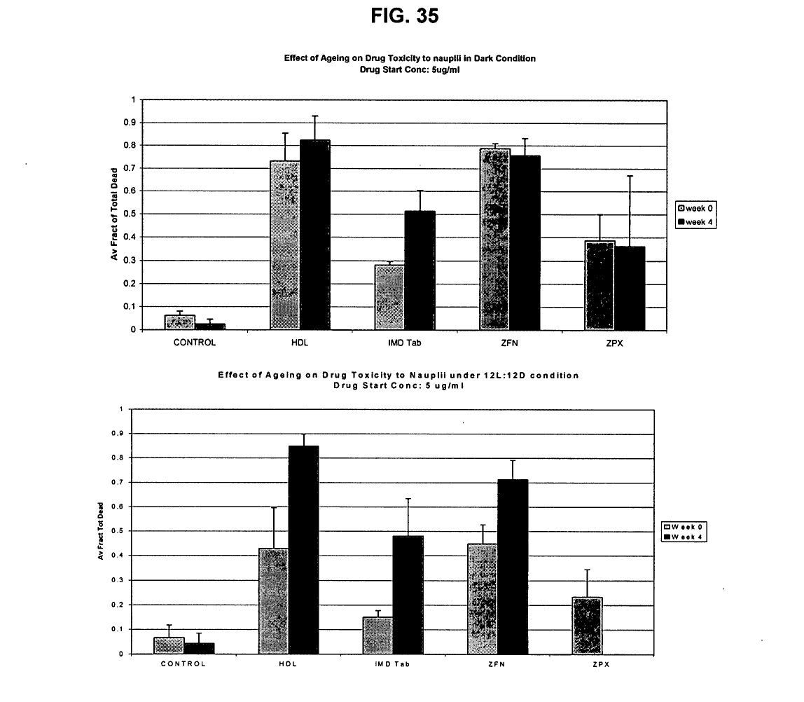

Preliminary experiments were carried out to examine the chemical stability of the drugs. 25 ug ml⁻¹ seawater suspensions of the drugs were prepared, in 3 replicates per condition, 3 replicate tubes containing 15 ml per test tube per prepared for each treatment type. The treatments consisted of:
i. Dark condition: tubes were wrapped in aluminium foil and kept in the dark at all times
ii. 12L:12D condition, where the tubes were subject incubated in a plant growth chamber with light regime of 12 hours light and 12 hours dark.
iii. UV treated tubes were subject to UV for half an hour.
   1 ml of solution was drawn from each tube and diluted 5×, 25×, 125× and nauplii toxicity assays was carried out. Sets (1) & (2) were tested at week 0, 1, 2 and after 4 weeks. “UV” set is only assayed after UV treatment. The drugs HDL, IMD, ZPX, ZFN were tested from 16 Sep. 2004-15 Oct. 2004. Whilst PCZ, TFZ, PZC and ZLT were tested from 21 Sep. 2004-21 Oct. 2004. CYZ was not tested but other studies carried out by the group indicate that this compound does not breakdown under UV and remains stable after four months in seawater. The results are given in FIGS. 34-35.
   Effect of UV

Only ZFN showed a reduction in toxicity under UV (FIG. 34). These was some increase in toxicity for PZC and TFZ under UV. The other compounds did not show significant difference from control.

Effect of Ageing

In the dark condition, after 4 weeks, only PCZ and TFZ showed appreciable decrease in toxicity (FIG. 35). The IMD drug suspension had increased in toxicity. On the other hand, in the 12L:12D (12 h light and 12 h dark) condition, most of the compounds showed significant changes. PCZ, TFZ and ZPX showed marked decrease in toxicity. On the other hand, IMD, HDL and ZFN increased in toxicity, suggesting that the breakdown products may be more toxic than the original compounds. PZC did not show an appreciable change. Toxicity of ZLT was not altered by any of the treatments applied.

Discussion

The two compounds PCZ, TFZ are sensitive to light, and appear to be unstable in seawater, degrading to less toxic by-products after 4 weeks in seawater. The breakdown and chemistry of the other compounds in seawater is less clear. However these preliminary results indicate that, with the exception of ZLT, they are unstable in seawater and under UV. It is likely that from analogues of the above, it would be possible to select molecules that will degrade into benign molecules in the environment.

Activity of Compounds in Field Testing

### Example 14

Field Testing

Methodology

Rapid field assessment of novel antifouling coatings using rods was introduced in Rittschof et al. (1992.b). Fouling on rods occurs more rapidly than on flat panels, enabling rapid assessment of the performance of coatings. This method was employed as the drug compounds are not optimised for field applications, and hence it was necessary to employ a rapid method.

Rod Preparation

6 mm diameter glass rods were cut to 150 mm length and prepared prior to coating by baking in a furnace at 150° C. for 24 hours and then cooling in a dessicator.

Drug Preparation

All pharmaceuticals were obtained in commercially available form. All tablets were reduced to fine powder with a mortar and pestle, and capsules were opened to extract the contents. The amount of drug powder required to attain the desired concentrations for all tests was calculated from product dosage information and then measured by weight on a high-resolution electronic balance. This was added to the coating and mixed thoroughly prior to application to obtain the concentration of 25 ug active ingredient per ml of coating mix. No data was obtained for TFZ. TFZ is an analogue of the drug PCZ.

Coating of Test Rods

The commercial antifouling product used to benchmark field experiments was VC Offshore with Teflon® manufactured by International Coatings. VC Offshore with Teflon is sold as a set of 2 parts, consisting of: 2 litre of liquid coating matrix (#YBA 707) and approximately 790 g of a copper-teflon powder (#YBA 779).

VC Offshore coating, made up according to the above ratio, applied on rods remain foul-free for up to 3-4 months in Singapore waters. For our experiments, a reduced copper coating (“VC50”) was prepared by mixing approximately 20 g of #YBA779 into 100 ml of #YBA707. The prepared coating was then allowed to stand so that bubbles from the mixing process dissipated. 15 ml centrifuge tubes were filled with 10 mls of prepared coating. Prepared glass rods were then immersed in the coating to a height of 100 mm and then suspended on a frame to air dry until coating cured. VC50 coated rods remain foul-free for approximately 6-8 weeks in Singapore waters.

Field Testing

Five replicate rods were tested for every experimental treatment, including controls. Two sets of controls were used, an uncoated blank control and a VC50 control without pharmaceutical additives. Rod were randomly arrayed on plastic vexar mesh attached to PVC frames and suspended at a depth of 10 cm from the water surface. All tests were conducted at the TMSI Biofouling experimental platform berthed at the Republic of Singapore Yacht Club (1° 17′ 40″ N, 103° 45′ 37.6″ E). Rods were examined visually weekly for the duration of testing, which typically lasts between 6 to 10 weeks. All fouling was scored as percentage cover of each category within a 6 mm×50 mm transect on each rod.

Results

In general, VC50 was found to have relatively poor performance against spirorbid polychaetes; percentage cover of this spirorbids on VC50 rods were consistently higher than that on uncoated blanks. This is attributed to two factors; one, spirorbid cover is generally low because of competition for space with barnacles, and two, few antifoulants direct target this group. Spirorbid cover therefore tends to be high when barnacles are prevented from settling, but IMD, ZFN, ZPX, HDL were found to exclude spirorbid settlement as well. However, only spirorbid cover on IMD and ZFN-treated rods were significantly less than blank as well as VC50 controls.

IMD

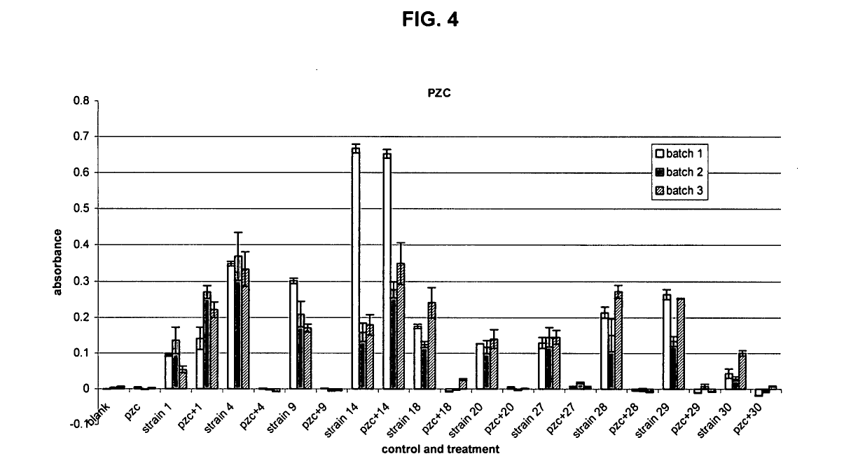

IMD significantly increased the fouling resistance of the base coating (p<0.001, Table 13). Average cover of hard fouling on uncoated blanks was 98%. At 55%, the cover of hard fouling on untreated VC50 was significantly lower, accounting for a 43% decrease compared to blank controls. IMD-treated VC50 coatings only had 0.67% cover of hard fouling (Table 13). This was significantly less than both blank controls and VC50 controls, with a reduction of 97.3% and 54.3% of the total hard fouling cover on blank controls and VC50 controls respectively (Table 13, FIG. 4.1). IMD demonstrated activity against all components of hard fouling (Table 13). Barnacle cover on IMD-treated coatings was significantly less than on both controls. IMD also proved highly resistant to settlement by spirorbid polychaetes, which VC50 performed poorly against. Cover of spirorbid polychaetes on VC50 control coatings was significantly higher than that on uncoated blanks, but VC50+IMD coatings had no settlement by spirorbids (Table 13, FIG. 4.1).

TABLE 13
Summary of t-tests comparing levels of hard fouling, barnacles, serpulids and spirorbid
on rods coated with IMD + VC50, VC50 and blank controls

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | p | (%) | t | p | (%) | t | p |
| Control | 98 | | | | 93 | | | 3.33 | | | 1.67 | | |
| VC50 | 55 | | | | 30 | | | 1.67 | | | 23.33 | | |
| IMD | 0.67 | | | | 0.67 | | | 0 | | | 0 | | |
| VC50 vs | −43 | 4 | −7.2 | <0.001 | −63 | −12.1 | <0.001 | −1.66 | −0.44 | NS | +25 | 4.6 | <0.01 |
| control | | | | | | | | | | | | | |
| IMD vs | −97.3 | 4 | −58.4 | <0.001 | −92.3 | −20.93 | <0.001 | −3.33 | −1 | NS | −1.67 | −1 | NS |
| control | | | | | | | | | | | | | |
| IMD vs | −54.3 | 4 | −9.35 | <0.001 | −29.3 | −9.9 | <0.001 | −1.67 | −1 | NS | −23.33 | −5.3 | <0.01 |
| VC50 | | | | | | | | | | | | | |

ZFN

Coatings containing ZFN increased the antifouling properties of VC50 coating significantly (p<0.01, Table 14). VC50 alone decreased cover of hard fouling from levels recorded on blank controls, with 55% cover compared to 98%. The addition of ZFN further decreased pressure by hard foulers, with only 10% total hard fouling recorded (Table 14, FIG. 4.1). For all categories of hard fouling other than serpulid polychaetes, ZFN significantly enhanced the performance of VC50 in reducing settlement (Table 14), but this was due in part to relatively low serpulid settlement throughout this set of trials (3.33% on blank controls). The addition of ZFN to VC50 accounted for a further decrease of 26.3% barnacle cover (Table 14, FIG. 4.1) and furthermore, 18.7% less spirorbid cover than VC50 (Table 14, FIG. 4.1).

TABLE 14
Summary of t-tests comparing levels of hard fouling, barnacles, serpulids and
spirorbids on rods coated with ZFN + VC50, VC50 and blank controls

| | | | |
|---|---|---|---|
| Total hard fouling | Barnacles | Serpulids | Spirorbids |

| | Mean | | | | Mean | | | Mean | | | Mean | | |
|---|---|---|---|---|---|---|---|---|---|---|---|---|---|
| | cover | | | | cover | | | cover | | | cover | | |
| Factor | (%) | df | t | p | (%) | T | p | (%) | t | p | (%) | t | p |
| Control | 98 | | | | 93 | | | 3.33 | | | | | |
| VC50 | 55 | | | | 30 | | | 1.67 | | | 23.33 | | |
| ZFN | 10 | | | | 3.67 | | | 1.67 | | | 4.67 | | |
| VC50 vs | −43 | 4 | −7.2 | <0.001 | −63 | −12.1 | <0.001 | −1.66 | −0.44 | NS | +21.66 | 4.6 | <0.01 |
| control | | | | | | | | | | | | | |
| ZFN vs | −88 | 4 | −26.94 | <0.001 | 89.33 | −19.6 | <0.001 | −1.66 | −0.44 | NS | +3 | 0.9 | NS |
| control | | | | | | | | | | | | | |
| ZFN vs | −45 | 4 | −6.97 | <0.01 | −26.33 | −8.3 | <0.001 | 0 | 0 | NS | −18.66 | −3.53 | <0.05 |
| VC50 | | | | | | | | | | | | | |

ZPX

The addition of ZPX to VC50 significantly improved performance against total hard fouling (17.3% compared to 98% on blank controls and 55% on VC50 controls). Reductions of hard fouling cover compared to VC50 were only significant for barnacles (Table 15, FIG. 4.1). ZPX also increased the resistance of VC50 to spirorbid fouling, however, this was non-significant, and spirorbid fouling on VC50+ZPX rods were still higher than on blank controls (Table 15).

TABLE 15
Summary of t-tests comparing levels of hard fouling, barnacles, serpulids and
spirorbids on rods coated with ZPX + VC50, VC50 and blank controls

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | p | (%) | t | p | (%) | t | p |
| Control | 98 | | | | 93 | | | 3.33 | | | 1.67 | | |
| VC50 | 55 | | | | 30 | | | 1.67 | | | 23.33 | | |
| ZPX | 17.3 | | | | 6.67 | | | 0 | | | 10.67 | | |
| VC50 | −43 | 4 | −7.2 | <0.001 | −63 | −12.1 | <0.001 | −1.66 | −0.44 | NS | +21.66 | 4.6 | <0.01 |
| vs | | | | | | | | | | | | | |
| control | | | | | | | | | | | | | |
| ZPX vs | −80.7 | 4 | −19.13 | <0.001 | −86.3 | −18.5 | <0.001 | −3.33 | −1 | NS | +9 | 1.64 | NS |
| control | | | | | | | | | | | | | |
| ZPX vs | −37.7 | 4 | −5.39 | <0.01 | −23.3 | −7 | <0.01 | −1.67 | −1 | NS | −12.66 | −1.86 | NS |
| VC50 | | | | | | | | | | | | | |

HDL

The percentage cover of total hard fouling was significantly less on VC50+HDL treated rods (30%) compared to VC50 controls (55%) (Table 16, FIG. 4.1). Serpulids were completely excluded on rods with VC50+HDL, but overall serpulid settlement was so low across all treatments that this did not result in statistically significant treatment effects. While barnacle cover on VC50+HDL rods was significantly less than on blank controls, the addition of HDL did not significantly reduce barnacle cover compared to VC50 controls (Table 4.4). The addition of HDL did, however, significantly improve the performance of VC50 against spirorbid polychaetes (Table 16).

TABLE 16
Summary of t-tests comparing levels of hard fouling, barnacles, serpulids and
spirorbids on rods coated with HDL + VC50, VC50 and blank controls.

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | p | (%) | t | p | (%) | t | p |
| Control | 98 | | | | 93 | | | 3.33 | | | 1.67 | | |
| VC50 | 55 | | | | 30 | | | 1.67 | | | 23.33 | | |
| HDL | 30 | | | | 23.3 | | | 0 | | | 6.67 | | |
| VC50 | −43 | 4 | 7.2 | <0.01 | −63 | 12.05 | <0.001 | −1.66 | 0.45 | NS | +21.66 | −4.6 | <0.05 |
| vs | | | | | | | | | | | | | |
| control | | | | | | | | | | | | | |
| HDL vs | −68 | 4 | 11.4 | <0.001 | −69.7 | 9.38 | <0.001 | −3.33 | 1 | NS | +5 | −2.12 | NS |
| control | | | | | | | | | | | | | |
| HDL vs | −25 | 4 | 3.06 | <0.05 | −6.7 | 1 | NS | −1.67 | 1 | NS | −16.66 | 3.54 | <0.05 |
| VC50 | | | | | | | | | | | | | |

CYZ

The addition of CYZ at a concentration of 25 μg/ml marginally increased the performance of VC50 coating. Levels of total hard fouling on CYZ-treated rods were 47.2% lower than that on rods with just the VC50 coating, but were only close to statistical significance (Table 17). Both VC50 and CYZ+VC50 significantly reduced hard fouling on the rods compared to untreated controls. Levels of barnacle fouling on CYZ-treated rods were significantly lower than both blank and VC50 controls (Table 17). The addition of CYZ also significantly enhanced the performance of VC50 against serpulid polychaetes.

TABLE 17
Summary of t-tests comparing levels of hard fouling, barnacles, serpulids and
spirorbids on rods coated with CYZ + VC50, VC50 and blank controls

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | p | (%) | t | p | (%) | t | p |
| Control | 100 | | | | 87.67 | | | 6.67 | | | 5.67 | | |
| VC50 | 41.7 | | | | 23.33 | | | 1.67 | | | 16.67 | | |
| CYZ | 22.0 | | | | 10.33 | | | 0 | | | 11.67 | | |
| VC50 | −58.3 | 4 | −6.29 | <0.01 | −64.34 | −10.4 | <0.001 | −5 | −2.12 | NS | +11 | 2.20 | <0.05 |
| vs | | | | | | | | | | | | | |
| control | | | | | | | | | | | | | |
| CYZ vs | −78.0 | 4 | −51.03 | <0.001 | −77.34 | −51.9 | <0.001 | −6.67 | −4 | <0.01 | +6 | 2.09 | NS |
| control | | | | | | | | | | | | | |
| CYZ vs | −19.7 | 4 | −2.09 | <0.05 | −13 | −2.16 | <0.05 | −1.67 | −1 | <0.05 | −5 | −1.06 | NS |
| VC50 | | | | | | | | | | | | | |

PZC

The addition of PZC to VC50 did not significantly improve antifouling performance against total hard fouling or any of the individual categories, although percentage cover of total hard foulers were ˜24% lower compared to VC50 alone (Table 18).

TABLE 18
Summary of t-tests comparing levels of hard fouling, barnacles, serpulids and
spirorbids on rods coated with PZC + VC50, VC50 and blank controls

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | P | (%) | t | p | (%) | t | p |
| Control | 100 | | | | 87.67 | | | 6.67 | | | 5.67 | | |
| VC50 | 41.7 | | | | 23.33 | | | 1.67 | | | 16.67 | | |
| PZC | 18.33 | | | | 9.33 | | | 0 | | | 9 | | |
| VC50 | −58.3 | 4 | −6.29 | <0.01 | −63.34 | −10.4 | <0.001 | −5 | −2.12 | NS | +11 | 2.20 | <0.05 |
| vs | | | | | | | | | | | | | |
| control | | | | | | | | | | | | | |
| PZC vs | −81.67 | 4 | 40.28 | <0.001 | −78.34 | 49.00 | <0.001 | −6.67 | 4.00 | <0.05 | +3.33 | −1.07 | NS |
| control | | | | | | | | | | | | | |
| PZC vs | −23.37 | 4 | 2.47 | NS | −14 | 2.32 | NS | −1.67 | 1.67 | NS | −7.67 | 16.67 | NS |
| VC50 | | | | | | | | | | | | | |

PCZ

The addition of PCZ significantly reduced total hard fouling compared to blank controls. Although total hard fouling cover on PCZ+VC50 coated rods was 16% lower than that on VC50 alone, this reduction was not statistically significant (Table 19). The analogue, TFZ was not tested.

TABLE 19
Summary of t-tests comparing levels of hard fouling, barnacles, serpulids and
spirorbids on rods coated with PCZ + VC50, VC50 and blank controls

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | p | (%) | t | p | (%) | t | p |
| Control | 98 | | | | 93 | | | 3.33 | | | 1.67 | | |
| VC50 | 55 | | | | 30 | | | 1.67 | | | 23.33 | | |
| PCZ | 39 | | | | 22.33 | | | 0 | | | 16.67 | | |
| VC50 | −43 | 4 | 7.2 | <0.01 | −63 | 12.05 | <0.001 | −1.66 | 0.45 | NS | +21.66 | −4.6 | <0.05 |
| vs | | | | | | | | | | | | | |
| control | | | | | | | | | | | | | |
| PCZ vs | −58 | 4 | 6.74 | <0.01 | −70.67 | 7.15 | <0.01 | −3.33 | 1 | NS | +15 | −1.59 | NS |
| control | | | | | | | | | | | | | |
| PCZ vs | −16 | 4 | 1.54 | NS | −7.67 | 0.82 | NS | −1.67 | 1 | NS | −6.66 | 0.65 | NS |
| VC50 | | | | | | | | | | | | | |

ZLT

The addition of ZLT did not significantly improve the antifouling performance of VC50. The cover of total hard fouling was reduced by 3.3% (Table 20). While the addition of ZLT caused 11.6% reduction in barnacle cover, rods coated with ZLT+VC50 actually had higher cover of serpulid and spirorbid polychaetes compared to VC50 alone.

TABLE 20
Summary of t-tests comparing levels of hard fouling, barnacles, serpulids and
spirorbids on rods coated with ZLT + VC50, VC50 and blank controls

| | Total hard fouling | | | | Barnacles | | | Serpulids | | | Spirorbids | | |
|---|---|---|---|---|---|---|---|---|---|---|---|---|---|
| | Mean | | | | Mean | | | Mean | | | Mean | | |
| | cover | | | | cover | | | cover | | | cover | | |
| Factor | (%) | df | t | p | (%) | T | p | (%) | t | p | (%) | t | p |
| Control | 98 | | | | 93 | | | 3.33 | | | 1.67 | | |
| VC50 | 55 | | | | 30 | | | 1.67 | | | 23.33 | | |
| ZLT | 51.67 | | | | 18.33 | | | 3.33 | | | 30 | | |
| VC50 | −43 | 4 | 7.2 | <0.01 | −63 | 12.05 | <0.001 | −1.66 | 0.45 | NS | +21.66 | −4.6 | <0.05 |
| vs | | | | | | | | | | | | | |
| control | | | | | | | | | | | | | |
| ZLT vs | −46.33 | 4 | 6.24 | <0.01 | −74.67 | 9.37 | <0.001 | 0 | 0 | NS | +28.33 | −3.62 | <0.05 |
| control | | | | | | | | | | | | | |
| ZLT vs | −3.33 | 4 | 0.36 | NS | −11.67 | 1.61 | NS | +1.66 | −0.45 | NS | +6.67 | −0.76 | NS |
| VC50 | | | | | | | | | | | | | |

### Experiment 15

Additional Field Data for IMD ZPX and ZFN

Two concentrations of each drug IMD, ZPX and ZFN in VC50 coating was prepared: one mixture with high drug dose (H=250 ug per ml of paint) and low drug dose (L=25 ug per ml of paint), for the following drugs: IMD, ZFN and ZPX.

The treatments tested were:

| | | Drug Composition |
|---|---|---|
| | | (concentration in |
| Type | Treatment | ug/ml) |
| Control - coating | VC50 | No drug additives |
| Single drug coating | IMD-HH | IMD(250) |
| | IMD-HL | IMD(137.5) |
| | IMD-LL | IMD(25) |
| | ZFN-HH | ZFN(250) |
| | ZFN-HL | ZFN(137.5) |
| | ZFN-LL | ZFN(25) |
| | ZPX-HH | ZPX(250) |
| | ZPX-HL | ZPX(137.5) |
| | ZPX-LL | ZPX(25) |
| Combinations: HH | IMD-H + ZFN-H | IMD(250) + ZFN(250) |
| | IMD-H + ZPX-H | IMD(250) + ZPX(250) |
| | ZFN-H + ZPX-H | ZFN(250) + ZPX(250) |
| Combinations: LL | IMD-L + ZFN-L | IMD(25) + ZFN(25) |
| | IMD-L + ZPX-L | IMD(25) + ZPX(25) |
| | ZFN-L + ZPX-L | ZFN(25) + ZPX(25) |
| Combinations: HL | IMD-L + ZFN-H | IMD(25) + ZFN(250) |
| | IMD-L + ZPX-H | IMD(25) + ZPX(250) |
| | ZFN-L + ZPX-H | ZFN(25) + ZPX(250) |
| | IMD-H + ZFN-L | IMD(250) + ZFN(25) |
| | IMD-H + ZPX-L | IMD(250) + ZPX(25) |
| | ZFN-H + ZPX-L | ZFN(250) + ZPX(25) |

Results & Discussion

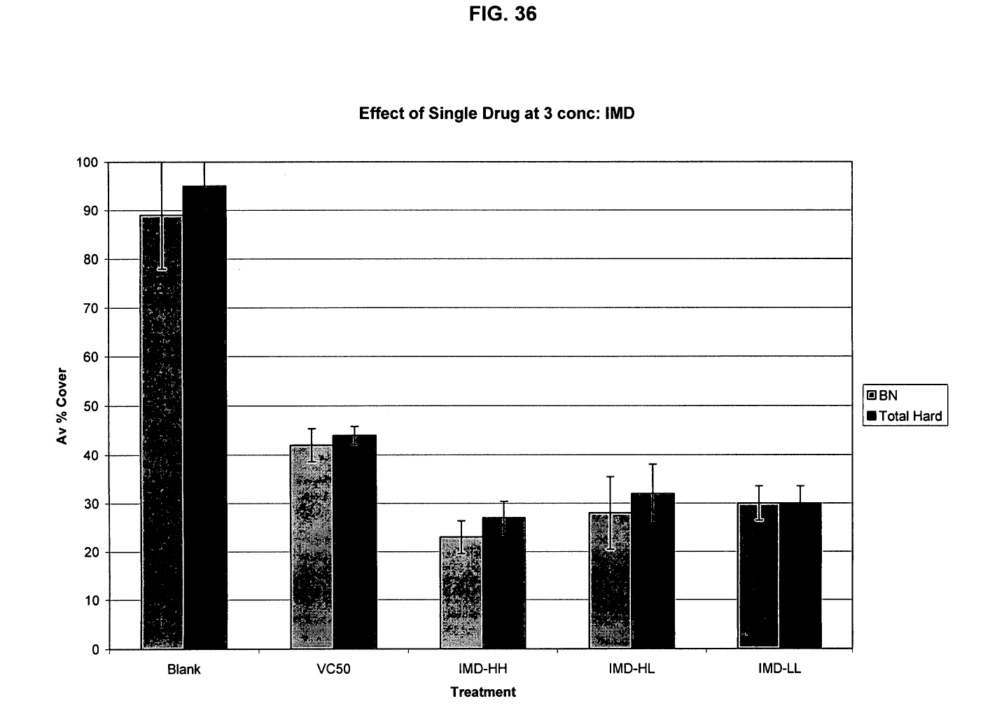

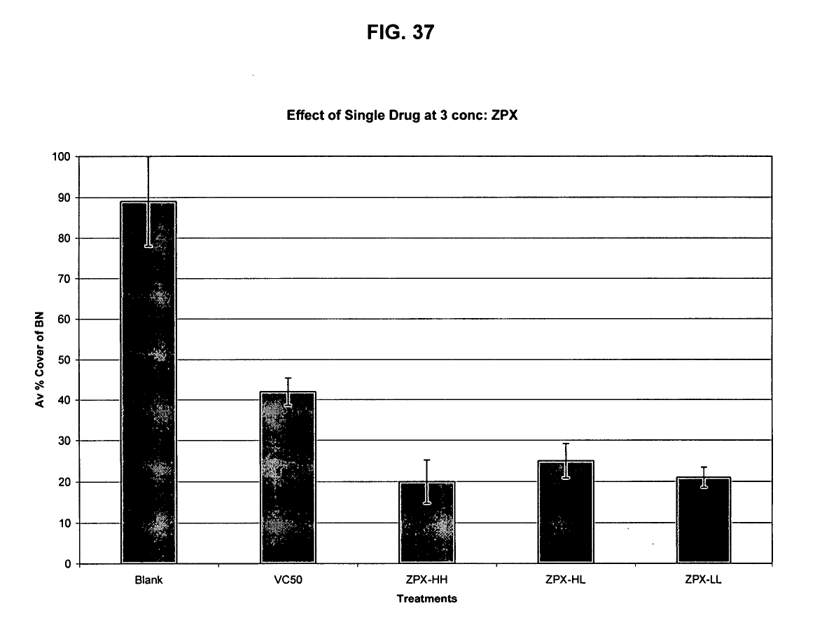

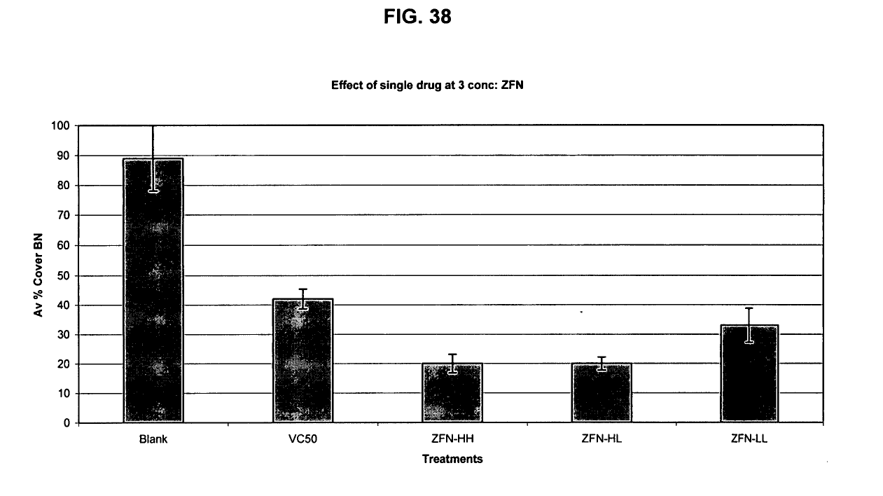

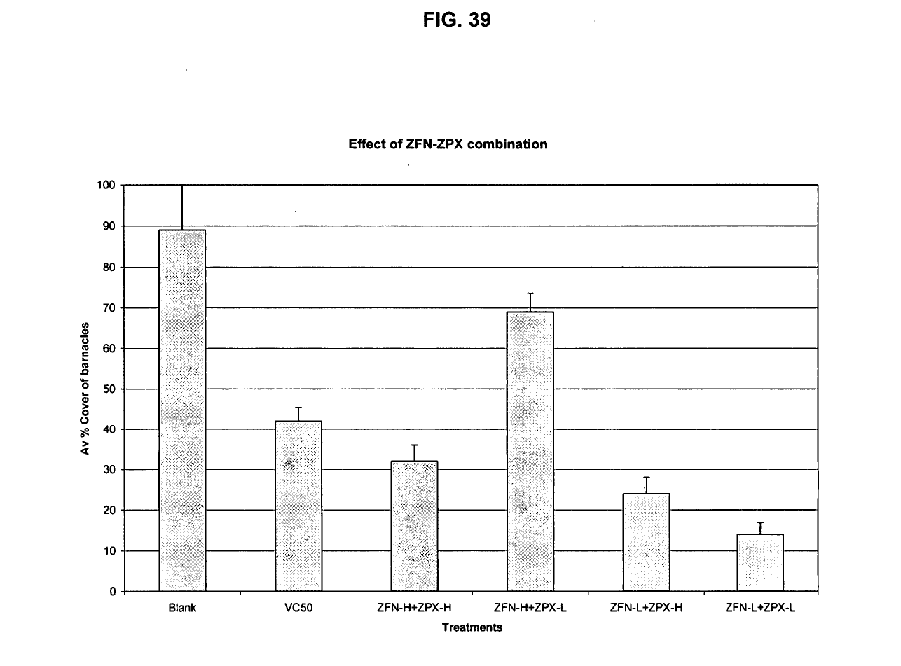

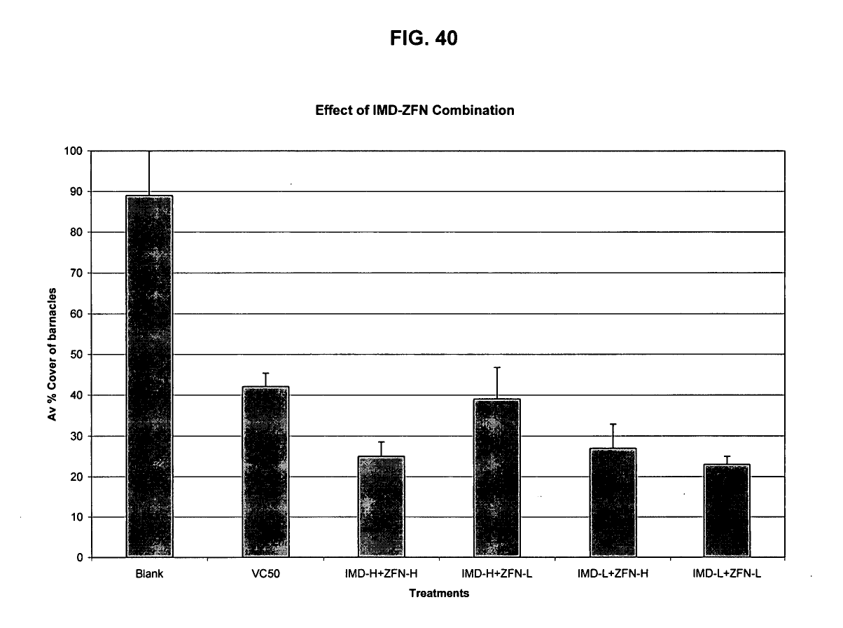

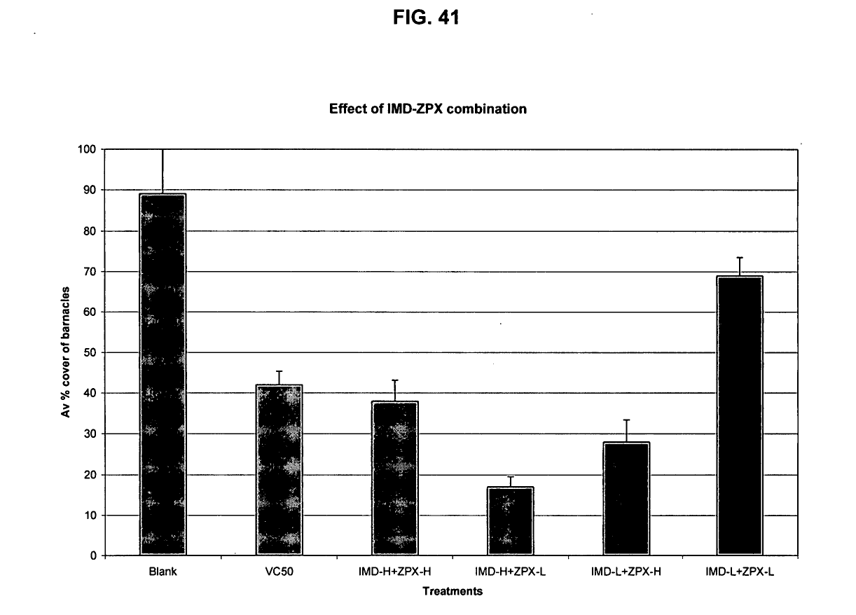

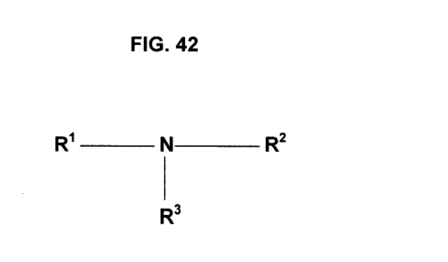

The results are given in FIGS. 36-41. The 3 drugs improved the performance of VC50 (FIGS. 36-38) at the 25 ug ml⁻¹ concentration, but increasing the amount of drug powder added did not improve the performance. The best performance was obtained with the low concentration mix of ZPX and ZFN, both at 25 ug ml⁻¹ (FIG. 39). What is evident from the results is that the above compounds, in their crude form, are still able to exert an antifouling effect even under high fouling pressures in natural field conditions.

### CONCLUSION

The drugs CYZ, IMD, ZPX, ZFN and HDL significantly improved the performance of VC50 coating, and demonstrated antifouling effects in field tests.

### GENERAL DISCUSSION

According to the embodiments reported in the Examples, the present invention provides the use of specific pharmaceuticals developed for human medical applications as antifoulants. This has a spectrum of benefits for navigating the technical and regulatory maze that leads to commercial products. Unlike the concept for development of novel natural products, pharmaceuticals enable faster, more efficient and potentially safe development for antifouling purposes as much is already known about these compounds. Examples include probable mechanism of action, toxicity in humans and other vertebrates, metabolic breakdown pathways, and chemical properties and reactivity. This information is important for human health and safety issues and a starting point for the process of obtaining registration from environmental regulatory agencies. The chemical properties, including structure and method of synthesis of compound are important for practical development of coatings. As the chemistry and primary mechanism of action of pharmaceuticals in vertebrates are known, the development process required to develop effective antifouling products from these compounds is very different from that for novel compounds. The existing drug information makes it possible to make informed decisions early in the process and will significantly expedite product development.

Table 21 is a summary of the bioactivities of the different compounds toward bacteria, larvae and organisms in the field.

TABLE 21
Summary of bio-activities of the compounds

| | Anti- | | | | | |
|---|---|---|---|---|---|---|
| | biotic | | | Prevention | Prevention | Overall |
| | activity | Prevention | Toxicity | of | of | perfor- |
| | against | of | to | settlement | settlement | mance |
| | marine | formation | barnacle | of barnacle | of Bugula | in field |
| Drug | bacteria | of biofilm | nauplii | cyprids | larvae | assay |
| CYZ | 8 | 8 | 3 | 4 | 5 | 5 |
| HDL | 8 | 6 | 6 | 3 | 4 | 4 |
| IMD | 8 | 7 | 5 | 8 | 7 | 1 |
| PCZ/ | 3 | 4 | 2 | 2 | 2 | 7 |
| TFZ | | | | | | |
| PZC | 2 | 5 | 4 | 7 | 3 | 6 |
| ZFN | 8 | 2 | 7 | 5 | 8 | 2 |
| ZLT | 1 | 3 | 1 | 6 | 1 | 8 |
| ZPX | 8 | 1 | 8 | 1 | 6 | 3 |

The activities of the compounds were crudely ranked from best (1) to worst performer (8)

CYZ, PZC and HDL show moderate toxic activity toward invertebrate larvae and this may be reflected in their field antifouling effect.

IMD prevented fouling in the field assay. PCZ and ZLT were essentially strong antibiotic compounds, toxic to bacteria and larvae and may be considered potential water treatment biocides. ZFN and ZPX are unusual in having poor toxicity but are able to reduce biofilm formation and prevent larval settlement.

The present inventors discovered strong antifouling activity in the compounds of the present invention. Although these are crude extracts, the activities detected indicate potential for further development.

CYZ: This compound is very toxic to marine larvae. There is evidence of activity against macrofoulers in the field. The compounds is relatively stable in seawater.

HDL: As in the case of CYZ, this compound is toxic to marine larvae.

PZC: This compound is moderately toxic to larvae and shows some antifouling effects in the field. It is bactericidal.

PCZ, TFZ are bactericides and toxic to larvae.

ZLT: This compound is a powerful marine bactericide and very toxic to marine larvae.

IMD: Evidence for IMD lies entirely on field performance data obtained to date. It remains the best performer in all field experiments.

ZPX: This compound is promising. It has shown very unusual activity in repelling fouling both on bacterial and macrofouler levels.

ZFN: Evidence to date consists of field antifouling performance and unusual activity against biofilm formation

### REFERENCES

Absolom, D. R., Lamberti, F. V., Policova, Z., Zingg, W. van Oss, C. J. and Newmann, A. W. 1983. Surface thermodynamics of bacterial adhesion. Applied and Environmental Microbiology 46:90-97.
Clare, A. S., D. Rittschof, D. J. Gerhart and J. S. Maki. 1992. Molecular approaches to non-toxic antifouling. Invertebrate Reproduction and Development Volume 22(1-3), pp. 67-76.
Clare, A. S. 1996. Marine natural product antifoulants: Status and potential. Biofouling 9: 211-229.
Dexter, S. C., Sullivan J. D. Jr., Williams, J. III and Watson, S. W. 1975. Influence of substrate wettability on the attachment of marine bacteria to various surfaces. Applied Microbiology 30:298-308.
Eli, L., H., Judes, and M. Rosenberg 1989. Saliva-mediated inhibition on promotion of bacterial adhesion to polystyrene. Biofouling 1:203-211.
Gerhart, D. J., Rittschof, D., Hooper, I. R., Eisenman, K., Meyer, A. E., Baier, R. E. and Young C. 1992. Rapid and inexpensive quantification of the combined polar components of surface wettability: application to biofouling. Biofouling 5:251-259.
Gherna R L, 1994. Culture preservation. Methods for general and molecular bacteriology. USA. Pp. 278-292.
Huang S-Y, Michael G. Hadfield. 2003. Composition and density of bacterial biofilms determine larval settlement of the polychaete Hydroides elegans. Marine Ecology Progress Series 260: 161-172.
Humphries, M., J. F. Jaworzyn, J. B. Cantwell, and A., Eakin. 1987. The use of non-ionic ethoxylated and propoxylated surfactants to prevent the adhesion of bacteria to solid surfaces. FEMS Microbiol. Lett. 42:91-101.
Klotz, S. A., D. J. Drutz, and J. E., Zajic, 1985. Factors governing adherence of Candida species to plastic surfaces. Infect. Immun. 50:97-101.
Libermann H R, 1983. Estimating LD50 using the probit technique: a basic computer program. Drug Chem Toxicol 6:111-116.
Maki J S, D Rittschof, J D Costlow & R Mitchell, 1988. Inhibition of attachment of larval barnacles, Balanus amphitrite, by bacterial surface films. Mar Biol. 97: 199-206.
Maki J S, L Ding, J Stokes, J H Kavouras & D Rittschof, 2000. Substratum/bacterial interactions and larval attachment: films and exopolysaccharides of Halomonas marina (ATCC 25374) and their effect on barnacle cypris larvae, Balanus amphitrite Darwin. Biofouling 16: 159-170.
Neeser, J. R., A. Chambaz, S. W. Vedovo, M.-J. Prigent, and B. Guggenheim. 1988. Specific and non-specific inhibition of adhesion of oral actinomyces and streptococci to erythrocytes and polystyrene by caseinoglycopeptide derivatives. Immun. Infect. 56:3201-3208.
O'Connor, N. J., and Richardson, D. L. 1996. Effects of bacterial films on attachment of barnacle (Balanus improvisus Darwin) larvae: laboratory and field studies. Journal of Experimental Marine Biology and Ecology 206:69-81.
Pringle, J. H. and M. Fletcher 1988. Influence of substratum hydration and adsorbed macromolecules on bacterial attachment to surfaces. Appl. Environ. Microbiol. 51:1321-1325.
Rittschof D, A S Clare, D J Gerhart, Sr Avelin Mary & J Bonaventura, 1992(a). Barnacle in-vitro assays for biologically active substances: toxicity and settlement inhibition assays using mass cultured Balanus amphitrite amphitrite Darwin. Biofouling 6: 115-122.
Rittschof D, A S Clare, D J Gerhart, J Bonaventura, C Smith & M G Hadfield, 1992(b). Rapid field assessment of antifouling and foul-release coatings. Biofouling 6: 181-191.
Rittschof D, 2000. Natural product antifoulants: one perspective on the challenges related to coatings development. Biofouling 15: 199-127.
Rittschof D, 2001. Natural product antifoulants and coatings development. In: McClintock J, Baker P (eds) Marine Chemical Ecology, CRC Press, NY, pp 543-557.
Rittschof Dan, Chien-Houng Lai, Lai-Mun Kok & Serena Lay-Ming Teo (2003). Pharmaceuticals as antifoulants: concepts and principles. Biofouling Vol. 19 (Supplement), pp. 207-212.
Shea, C. and Williamson, J. C. 1990. Rapid analysis of bacterial adhesion in a microplate assay. Biotechniques 8(6):610-611.
Teo Serena Lay-Ming, Choong Mei Fun & Dan Rittschof (2003). Pharmaceuticals as antifoulants. Paper Presentation. Proceedings, HSA-NUS Joint Scientific Seminar 9 Apr. 2003, pp. 106-111.
Willemsen P R, K Overbeke, A Suurmond, 1998. Repetitive testing of TBTO, Sea-Nine 211 and farnesol using Balanus amphitrite (Darwin) cypris larvae: variability in larval sensitivity. Biofouling 12: 133-147.

What is claimed is:

1. A biocidal and/or biostatic composition, the composition comprising at least one compound having the formula R¹R²NR³, wherein R¹, R² and R³, are selected from the group consisting of hydrido, alkyl, aryl, heteroaryl, cycloalkyl, cycloaryl, cycloheteroaryl, alkenyl and alkynyl.

2. The composition according to claim 1, wherein the at least one compound is a substituted amine wherein R¹ and R² are selected from the group consisting of alkyl, heteroaryl, cycloalkyl, cycloaryl, cycloheteroalkyl, and cycloheteroaryl; and R³ is alkyl.

3. The composition according to claim 2, wherein R¹ and R² form part of a carbocyclic or heterocyclic ring.

4. The composition according to claim 2, wherein R1 and R2 form part of a carbocyclic or heterocyclic ring; and wherein R3 is selected from the group consisting of methyl, ethyl, propyl and butyl.

5. The composition according to claim 1, wherein the at least one compound is selected from the group consisting of: cyclizine, haloperidol decanoate, loperamide, prochloroperazine, trifluoperazine, fluoxetine, ondansetron, sertraline hydrochloride, olanzapine and salt(s) thereof.

6. The composition according to claim 1, wherein the composition is for prevention of fouling.

7. The composition according to claim 1, wherein the composition is for prevention of biofilm formation.

8. A method for biocidal and/or biostatic treatment of a medium comprising:
providing at least one compound having the formula R¹R²NR³, wherein R¹, R² and R³ are selected from the group consisting of hydrido, alkyl, aryl, heteroaryl, cycloalkyl, cycloaryl, cycloheteroaryl, alkenyl and alkynyl; and
applying the compound to the medium.

9. The method according to claim 8, wherein the at least one compound is a substituted amine wherein R¹ and R² are selected from the group consisting of alkyl, heteroaryl, cycloalkyl, cycloaryl, cycloheteroalkyl, and cycloheteroaryl; and R³ is alkyl.

10. The method according to claim 9, wherein R¹ and R² form part of a carbocyclic or heterocyclic ring.

11. The method according to claim 9, wherein R¹ and R² form a part of carbocyclic or heterocyclic ring, and wherein R³ is selected from the group consisting of methyl, ethyl, propyl and butyl.

12. The method according to claim 8, wherein the at least one compound is selected from the group consisting of: cyclizine, haloperidol decanoate, loperamide, prochloroperazine, trifluoperazine, fluoxetine, ondansetron, sertraline hydrochloride, olanzapine and salt(s) thereof.

13. The method according to claim 8, wherein the medium is a solid medium, the method further comprising mixing the compound with a curable binder to form a mixture before applying the composition to the solid medium.

14. The method according to claim 13, wherein the mixture is allowed to cure.

15. The method according to claim 13, wherein the binder comprises at least one anti-fouling compound at less than standard concentration.

16. The method according to claim 8, wherein the treatment is for prevention of fouling of the medium.

17. The method according to claim 8, wherein the treatment is for prevention of biofilm formation.

18. A coated substrate wherein the coating comprises at least one compound having the formula R¹R²NR³, wherein R¹, R² and R³ are selected from the group consisting of hydrido, alkyl, aryl, heteroaryl, cycloalkyl, cycloaryl, cycloheteroaryl, alkenyl and alkynyl.

19. The coated substrate according to claim 18, wherein the at least one compound is a substituted amine wherein R¹ and R² are selected from the group consisting of alkyl, heteroaryl, cycloalkyl, cycloaryl, cycloheteroalkyl, and cycloheteroaryl; and R³ is alkyl.

20. The coated substrate according to claim 18, wherein the at least one compound is selected from the group consisting of: cyclizine, haloperidol decanoate, loperamide, prochlorperazine, trifluoperazine, fluoxetine, ondansetron, sertraline hydrochloride, and olanzapine.
